SEQUENCE LISTING (77 sequences)

SEQ ID NO: 1 - DNA, 20 bp, synthetic construct
  source 1..20: mol_type = other DNA; note = protospacer region
ctcgttgtccaggtaggccc

SEQ ID NO: 2 - DNA, 20 bp, synthetic construct
  source 1..20: mol_type = other DNA; note = protospacer region
tggaccaccagctcctgtag

SEQ ID NO: 3 - protein, 1368 aa, Streptococcus pyogenes [sequence omitted]

SEQ ID NO: 4 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1082 aa, Neisseria meningitidis
MAAFKPNPINYILGLDIGIASVGWAMVEIDEEENPIRLIDLGVRVFERAEVPKTGDSLAMVRRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHISGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 6 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 7 - DNA, 20 bp, synthetic construct
  source 1..20: mol_type = other DNA; note = protospacer
ctcgttgtccaggtaggccc

SEQ ID NO: 8 - DNA, 20 bp, synthetic construct
  source 1..20: mol_type = other DNA; note = protospacer
tggaccaccagctcctgtag

SEQ ID NO: 9 - DNA, 100 bp, synthetic construct
  source 1..100: mol_type = other DNA; note = ssODN
cttccatgcaggccctgggtggggccgggtctccccactgcagcctctcgttgtccaggtatggcccggatccactgccttccccgacttcaccaacccc

SEQ ID NO: 10 - DNA, 100 bp, synthetic construct
  source 1..100: mol_type = other DNA; note = ssODN
tgcccatcccccttgcaggttccccaaggactctagcacctggactgtggaccaccagctcctgtggggagaggccctgctcatcaccccagtgctccag

SEQ ID NO: 11 - DNA, 20 bp, synthetic construct
  source 1..20: mol_type = other DNA; note = primer
agctgctcattgacctccag

SEQ ID NO: 12 - DNA, 20 bp, synthetic construct
  source 1..20: mol_type = other DNA; note = primer
caatccacatgccgtcgaag

SEQ ID NO: 13 - DNA, 20 bp, synthetic construct
  source 1..20: mol_type = other DNA; note = primer
aattcagcctcttcctgtgc

SEQ ID NO: 14 - DNA, 20 bp, synthetic construct
  source 1..20: mol_type = other DNA; note = primer
catacgttcctctttccgcc

SEQ ID NO: 15 - DNA, 22 bp, synthetic construct
  source 1..22: mol_type = other DNA; note = primer
tgacaggtttccctcttcccag

SEQ ID NO: 16 - DNA, 22 bp, synthetic construct
  source 1..22: mol_type = other DNA; note = primer
ttgataacctacactgcggggg

SEQ ID NO: 17 - DNA, 18 bp, synthetic construct
  source 1..18: mol_type = other DNA; note = primer
agtggggcttccatgcag

SEQ ID NO: 18 - DNA, 20 bp, synthetic construct
  source 1..20: mol_type = other DNA; note = protein
ggttggtgaagtcggggaag

SEQ ID NO: 19 - DNA, 22 bp, synthetic construct
  source 1..22: mol_type = other DNA; note = primer
ccaaggactctagcacctggac

SEQ ID NO: 20 - DNA, 22 bp, synthetic construct
  source 1..22: mol_type = other DNA; note = primer
gggaagtagccagtcacttcgg

SEQ ID NO: 21 - DNA, 20 bp, synthetic construct
  source 1..20: mol_type = other DNA; note = sgRNA Target Sequence
tggaccaccagctcctgtag

SEQ ID NO: 22 - DNA, 20 bp, synthetic construct
  source 1..20: mol_type = other DNA; note = Off-target sequence
tagaccaccagctcctgcag

SEQ ID NO: 23 - DNA, 20 bp, synthetic construct
  source 1..20: mol_type = other DNA; note = Off-target sequence
ctcaccacctgctcctgtag

SEQ ID NO: 24 - DNA, 20 bp, synthetic construct
  source 1..20: mol_type = other DNA; note = Off-target sequence
ttgaccagcagctcctgtcg

SEQ ID NO: 25 - DNA, 20 bp, synthetic construct
  source 1..20: mol_type = other DNA; note = sgRNA Target Sequence
tggaccaccagctcctgtag

SEQ ID NO: 26 - DNA, 20 bp, synthetic construct
  source 1..20: mol_type = other DNA; note = Off-target sequence
ctggttgtccaggtgggccc

SEQ ID NO: 27 - DNA, 20 bp, synthetic construct
  source 1..20: mol_type = other DNA; note = Off-target sequence
ctcgatggccaggtaggcct

SEQ ID NO: 28 - DNA, 20 bp, synthetic construct
  source 1..20: mol_type = other DNA; note = Off-target sequence
attattgaccaggtaggccc

SEQ ID NO: 29 - DNA, 20 bp, synthetic construct
  source 1..20: mol_type = other DNA; note = primer
ccctcctctgtgtgccatta

SEQ ID NO: 30 - DNA, 21 bp, synthetic construct
  source 1..21: mol_type = other DNA; note = primer
gtgccatattttggggaccac

SEQ ID NO: 31 - DNA, 20 bp, synthetic construct
  source 1..20: mol_type = other DNA; note = primer
ggggcatggtcagatgatgg

SEQ ID NO: 32 - DNA, 22 bp, synthetic construct
  source 1..22: mol_type = other DNA; note = primer
cacagaaattcctgaggccaac

SEQ ID NO: 33 - DNA, 20 bp, synthetic construct
  source 1..20: mol_type = other DNA; note = primer
ggagaggctgaccttcatgg

SEQ ID NO: 34 - DNA, 20 bp, synthetic construct
  source 1..20: mol_type = other DNA; note = primer
tcgtgctttcctgaccatcg

SEQ ID NO: 35 - DNA, 20 bp, synthetic construct
  source 1..20: mol_type = other DNA; note = primer
agtgtgcttccactgtcgtt

SEQ ID NO: 36 - DNA, 20 bp, synthetic construct
  source 1..20: mol_type = other DNA; note = primer
gtgcgggtaaccttctccat

SEQ ID NO: 37 - DNA, 20 bp, synthetic construct
  source 1..20: mol_type = other DNA; note = primer
ttcctctgctgctgagttgg

SEQ ID NO: 38 - DNA, 20 bp, synthetic construct
  source 1..20: mol_type = other DNA; note = primer
gccgattaaaaggctgtcgc

SEQ ID NO: 39 - DNA, 20 bp, synthetic construct
  source 1..20: mol_type = other DNA; note = primer
agagccctggaggtcattgt

SEQ ID NO: 40 - DNA, 20 bp, synthetic construct
  source 1..20: mol_type = other DNA; note = primer
ctgtctggcctctgaatcgg

SEQ ID NO: 41 - DNA, 23 bp, synthetic construct
  source 1..23: mol_type = other DNA; note = sgRNA sequence
gaggagccactcagctctcaggg

SEQ ID NO: 42 - DNA, 23 bp, synthetic construct
  source 1..23: mol_type = other DNA; note = sgRNA sequence
atcgaagcggctgttgggggggg

SEQ ID NO: 43 - DNA, 23 bp, synthetic construct
  source 1..23: mol_type = other DNA; note = sgRNA sequence
ctggacgtgatgatggagacgag

SEQ ID NO: 44 - DNA, 23 bp, synthetic construct
  source 1..23: mol_type = other DNA; note = sgRNA sequence
agtggccggcgtatcagccgtgg

SEQ ID NO: 45 - DNA, 23 bp, synthetic construct
  source 1..23: mol_type = other DNA; note = sgRNA sequence
tgctggccacggccgatagccgg

SEQ ID NO: 46 - DNA, 23 bp, synthetic construct
  source 1..23: mol_type = other DNA; note = sgRNA sequence
aagccgcagacgtcggccgtcgg

SEQ ID NO: 47 - DNA, 23 bp, synthetic construct
  source 1..23: mol_type = other DNA; note = sgRNA sequence
caccagctcctgtaggggggagg

SEQ ID NO: 48 - DNA, 23 bp, synthetic construct
  source 1..23: mol_type = other DNA; note = sgRNA sequence
accaagggtggggaggcctgagg

SEQ ID NO: 49 - DNA, 23 bp, synthetic construct
  source 1..23: mol_type = other DNA; note = sgRNA sequence
taacacgatcgtgaattagctgg

SEQ ID NO: 50 - DNA, 30 bp, synthetic construct
  source 1..30: mol_type = other DNA; note = 2237G repair ssODN
gactgtggaccaccagctcctgtggggaga

SEQ ID NO: 51 - DNA, 30 bp, synthetic construct
  source 1..30: mol_type = other DNA; note = allele 1
gactgtggaccaccagctcctgtagggaga

SEQ ID NO: 52 - DNA, 30 bp, synthetic construct
  source 1..30: mol_type = other DNA; note = allele 2
gactgtggaccaccagctcctgtgggggga

SEQ ID NO: 53 - DNA, 33 bp, synthetic construct
  source 1..33: mol_type = other DNA; note = 1441 insT repair ssODN
gcctctcgttgtccaggtatggcccggatccac

SEQ ID NO: 54 - DNA, 33 bp, synthetic construct
  source 1..33: mol_type = other DNA; note = allele 2
gcctctcgttgtccaggtatggcccgggtccac

SEQ ID NO: 55 - DNA, 33 bp, synthetic construct; identical to SEQ ID NO: 54
  source 1..33: mol_type = other DNA; note = allele 1

SEQ ID NO: 56 - DNA, 30 bp, synthetic construct
  misc_feature 20: note = n is a, c, g, or t
  misc_feature 22: note = n is a, c, g, or t
  misc_feature 25: note = n is a, c, g, or t
  misc_feature 28: note = n is a, c, g, or t
  source 1..30: mol_type = other DNA; note = unedited allele
gcctctcgttgtccaggtangnccnggntc

SEQ ID NO: 57 - DNA, 30 bp, synthetic construct
  source 1..30: mol_type = other DNA; note = 1441 delT corrected
gcctctcgttgtccaggtatggcccggatc

SEQ ID NO: 58 - DNA, 28 bp, synthetic construct
  misc_feature 24: note = n is a, c, g, or t
  source 1..28: mol_type = other DNA; note = unedited allele
gactgtggaccaccagctcctgtngggg

SEQ ID NO: 59 - DNA, 28 bp, synthetic construct
  source 1..28: mol_type = other DNA; note = 2337G>A corrected
gactgtggaccaccagctcctgtgggag

SEQ ID NO: 60 - DNA, 20 bp, synthetic construct
  source 1..20: mol_type = other DNA; note = protospacer
ggaccaccagctcctgtagg

SEQ ID NO: 61 - DNA, 20 bp, synthetic construct
  source 1..20: mol_type = other DNA; note = protospacer
gcccaggaagccgcagacgt

SEQ ID NO: 62 - DNA, 20 bp, synthetic construct
  source 1..20: mol_type = other DNA; note = Protospacer
cagaggagctgtgtgtgcac

SEQ ID NO: 63 - DNA, 100 bp, synthetic construct; identical to SEQ ID NO: 10
  source 1..100: mol_type = other DNA; note = ssODN

SEQ ID NO: 64 - DNA, 100 bp, synthetic construct
  source 1..100: mol_type = other DNA; note = ssODN
agaaatcctgcagtttaacctgctgggggtgcctctggtcggggcagacgtctgtggcttcctgggcaacacctcagaggagctgtgtgtgcactggacc

SEQ ID NO: 65 - DNA, 100 bp, synthetic construct
  source 1..100: mol_type = other DNA; note = ssODN
gggccaacgtctgcggcttcctgggcaacacctcagaggagctatgtgtgcgctggacccagctgggggccttctaccccttcatgcggaaccacaacag

SEQ ID NO: 66 - DNA, 21 bp, synthetic construct
  source 1..21: mol_type = other DNA; note = primer
tcccattcatcacccgtatgc

SEQ ID NO: 67 - DNA, 20 bp, synthetic construct
  source 1..20: mol_type = other DNA; note = primer
aggtcgtaccatgtgcccaa

SEQ ID NO: 68 - DNA, 20 bp, synthetic construct
  source 1..20: mol_type = other DNA; note = primer
ctgaggaccagcctgactct

SEQ ID NO: 69 - DNA, 20 bp, synthetic construct
  source 1..20: mol_type = other DNA; note = primer
ccaccctaccagactgagca

SEQ ID NO: 70 - DNA, 33 bp, synthetic construct
  misc_feature 24: note = n is a, c, g, or t
  source 1..33: mol_type = other DNA; note = Unedited sequence, 1441delT corrected
gactgtggaccaccagctcctgtnggggggagg

SEQ ID NO: 71 - DNA, 32 bp, synthetic construct
  source 1..32: mol_type = other DNA; note = 2237G>A corrected, double corrected
gactgtggaccaccagctcctgtggggagagg

SEQ ID NO: 72 - DNA, 40 bp, synthetic construct
  source 1..40: mol_type = other DNA; note = 2237 OT-1
cctcccttcctagaccaccagctcctgcaggagggcttgg

SEQ ID NO: 73 - DNA, 40 bp, synthetic construct
  source 1..40: mol_type = other DNA; note = 2237 OT-2
gcccctgcctctacaggagcaggtggtgaggatggctccg

SEQ ID NO: 74 - DNA, 40 bp, synthetic construct
  source 1..40: mol_type = other DNA; note = 2237 OT-3
cacgctgccacgacaggagctgctggtcaactcctcctct

SEQ ID NO: 75 - DNA, 40 bp, synthetic construct
  source 1..40: mol_type = other DNA; note = 1441 OT-1
ccccgtatccctggttgtccaggtgggccctgggagaaca

SEQ ID NO: 76 - DNA, 40 bp, synthetic construct
  source 1..40: mol_type = other DNA; note = 1441 OT-2
cagctgccgtctcgatggccaggtaggcctgggcaaggac

SEQ ID NO: 77 - DNA, 40 bp, synthetic construct
  source 1..40: mol_type = other DNA; note = 1441 OT-3
aattaggctagggcctacctggtcaataatgaaataattg

(19) United States
(12) Patent Application Publication
(10) Pub. No.: US 2024/0076660 A1
(43) Pub. Date: Mar. 7, 2024

(54) GENE CORRECTION OF POMPE DISEASE AND OTHER AUTOSOMAL RECESSIVE DISORDERS VIA RNA-GUIDED NUCLEASES

(71) Applicant: Wisconsin Alumni Research Foundation, Madison, WI (US)

(72) Inventors: Krishanu Saha, Middleton, WI (US); Jared Matthew Carlson-Stevermer, Burlingame, CA (US); Lucille Katherine Kohlenberg, Elm Grove, WI (US)

(21) Appl. No.: 18/348,573

(22) Filed: Jul. 7, 2023

Related U.S. Application Data
(63) Continuation of application No. 16/674,448, filed on Nov. 5, 2019, now Pat. No. 11,739,320.
(60) Provisional application No. 62/755,980, filed on Nov. 5, 2018.

(51) Int. Cl. C12N 15/11; A61K 31/7088; A61K 35/545; A61P 3/00; C12N 9/22; C12N 15/86

(52) U.S. Cl. CPC C12N 15/11; A61K 31/7088; A61K 35/545; A61P 3/00; C12N 9/22; C12N 15/86; C12N 2310/20; C12N 2310/351; C12N 2800/80

(57) ABSTRACT

Described herein are methods of modifying a target gene in a patient or in a patient-derived cell, wherein the patient has an autosomal recessive disorder with compound heterozygous mutations, the methods including delivering a first modified guide RNA, a second modified guide RNA, a Cas9 polypeptide, a biotin-binding molecule, a first biotinylated donor polynucleotide, and a second biotinylated donor polynucleotide. The first modified guide RNA and the first biotinylated donor polynucleotide correct a first diseased allele, and the second modified guide RNA and the second biotinylated donor polynucleotide correct a second diseased allele.

### CROSS-REFERENCE TO RELATED APPLICATIONS

This application is a continuation of U.S. application Ser. No. 16/674,448, filed on Nov. 5, 2019, which claims priority to U.S. Provisional Application 62/755,980 filed on Nov. 5, 2018, which is incorporated herein by reference in its entirety.

### STATEMENT REGARDING FEDERALLY SPONSORED RESEARCH & DEVELOPMENT

This invention was made with government support under GM119644 awarded by the National Institutes of Health and CBET1350178 awarded by the National Science Foundation. The government has certain rights in the invention.

### SEQUENCE LISTING

The Instant Application contains a Sequence Listing which has been submitted electronically in XML format and is hereby incorporated by reference in its entirety. Said XML copy, created on Oct. 19, 2023, is named “Replacement_Seq_List-WIS0051USC” and is 89,815 bytes in size. The Sequence Listing does not go beyond the disclosure in the application as filed.

### FIELD OF THE DISCLOSURE

The present disclosure is related to compositions and methods for the treatment of autosomal recessive diseases.

### BACKGROUND

Infantile-onset Pompe disease is an autosomal recessive glycogen storage disorder cause by mutations in the acid-a-glucosidase (GAA) gene that encodes an enzyme that breaks down glycogen within the lysosome (FIG. 1a). Over 400 different GAA mutations have been noted within ClinVar, and detailed case studies indicate a buildup of lysosomic glycogen, leading to clinical complications, most prominently in cardiac and muscle tissues. Left untreated, patients with infantile-onset Pompe disease typically die within the first year of life, and Pompe disease is now frequently included within newborn screening panels. Enzyme replacement therapy (ERT) using recombinant human GAA (rhGAA) is currently the only approved clinical treatment for Pompe disease. However, patients require high levels of enzyme injected biweekly, rendering the treatment expensive and inconvenient. ERT may also be less effective in a subset of patients that are cross-reactive immunologic material (CRIM) negative.

Newer therapies in development for Pompe disease have primarily made use of integrated viral cassettes including transgenes to express GAA from exogenous promoters. Viral particles are injected either directly into muscle or administered systemically and transported to the liver. Direct injection to cardiac or skeletal muscle provided long term recovery of phenotype (10-fold reduction in glycogen content) to transduced cells but did not affect non-transduced cells and required high viral loads (>10¹⁰ viral genomes/kg). Silencing of the viral transgene, immune response to the viral vector, and insertional oncogenesis are outstanding concerns with these viral gene therapy approaches. Anti-sense oligonucleotides can be introduced to correct splicing in diseased patients that possessed mutations at splicing sites but would only be beneficial to a subset of potential patients. Finally, autologous cell therapy has been proposed using cells engineered to constitutively overexpress GAA. None of these approaches retain endogenous GAA regulation nor have corrected the underlying GAA mutations.

What is needed are novel strategies for correction of the mutated alleles Pompe disease and other autosomal recessive disorders which could be a promising in vivo somatic gene editing strategy or an alternative strategy to generate gene corrected cells ex vivo for cell therapy.

### BRIEF SUMMARY

In one aspect, a modified guide RNA comprises
a crRNA comprising, a single-stranded protospacer sequence and a first complementary strand of a binding region for the Cas9 polypeptide, and
a tracrRNA comprising a second complementary strand of the binding region for the Cas9 polypeptide,
wherein the crRNA or the tracrRNA comprises an aptamer that binds a biotin-binding molecule,
wherein the crRNA and the tracrRNA hybridize through the first and second complementary strands of the binding region for the Cas9 polypeptide,
wherein the single stranded protospacer region comprises

(SEQ ID NO: 1)
CTCGTTGTCCAGGTAGGCCC,
(SEQ ID NO: 2)
TGGACCACCAGCTCCTGTAG,
(SEQ ID NO: 60)
GGACCACCAGCTCCTGTAGG,
(SEQ ID NO: 61)
GCCCAGGAAGCCGCAGACGT,
or
(SEQ ID NO: 62)
CAGAGGAGCTGTGTGTGCAC.

In another aspect, a guide RNA comprises
a crRNA comprising, a single-stranded protospacer sequence and a first complementary strand of a binding region for the Cas9 polypeptide, and
a tracrRNA comprising a second complementary strand of the binding region for the Cas9 polypeptide,
wherein the crRNA and the tracrRNA hybridize through the first and second complementary strands of the binding region for the Cas9 polypeptide,
wherein the single stranded protospacer region comprises

(SEQ ID NO: 1)
CTCGTTGTCCAGGTAGGCCC,
(SEQ ID NO: 2)
TGGACCACCAGCTCCTGTAG,
(SEQ ID NO: 60)
GGACCACCAGCTCCTGTAGG,
(SEQ ID NO: 61)
GCCCAGGAAGCCGCAGACGT,
or
(SEQ ID NO: 62)
CAGAGGAGCTGTGTGTGCAC.

Also included herein are RNP complexes comprising the guide RNAs and modified guide RNAs, and a Cas9 polypeptide or active fragment thereof.

In another aspect, a method of modifying a target gene in a patient or in a patient-derived cell, wherein the patient has an autosomal recessive disorder with compound heterozygous mutations comprises
delivering to the patient or cell a first modified guide RNA, a second modified guide RNA, a Cas9 polypeptide, a biotin-binding molecule, a first biotinylated donor polynucleotide, and a second biotinylated donor polynucleotide, wherein each modified guide RNA comprises,

a crRNA comprising, a single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,

wherein the single-stranded protospacer sequence of the modified guide RNA hybridizes to a sequence in the target gene to be modified. wherein the first modified guide RNA and the first biotinylated donor polynucleotide
correct a first diseased allele,
wherein the second modified guide RNA and the second biotinylated donor polynucleotide correct a second diseased allele.

In an aspect, a method of treating a patient with an autosomal recessive disorder with compound heterozygous mutations comprises transplanting the cell made by the foregoing method.

In another aspect, a method of modifying a target gene in a patient or in a patient-derived cell, wherein the patient has Pompe disease, comprises
delivering to the patient or cell a first modified guide RNA, a second modified guide RNA, a Cas9 polypeptide, a biotin-binding molecule, a first biotinylated donor polynucleotide, and a second biotinylated donor polynucleotide, wherein each modified guide RNA comprises,

a crRNA comprising, a single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,

wherein the single-stranded protospacer sequence of the modified guide RNA hybridizes to a sequence in the target gene to be modified.
wherein the first modified guide RNA and the first biotinylated donor polynucleotide correct a first diseased allele,
wherein the second modified guide RNA and the second biotinylated donor polynucleotide correct a second diseased allele,
wherein the single stranded protospacer region of the first modified guide RNA comprises CTCGTTGTCCAGGTAGGCCC (SEQ ID NO: 1) and the single stranded protospacer region of the second guide RNA comprises TGGACCACCAGCTCCTGTA (SEQ ID NO: 2).

In another aspect, a method of making an RNP complex, comprises selecting a single stranded protospacer sequence by identifying a mutant allele to be corrected, wherein the mutant allele is within 0 to 100 bases of a protospacer adjacent motif,
producing a modified guide RNA comprising the selected single stranded protospacer region, wherein the modified guide RNA comprises

a crRNA comprising the single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,
selecting a donor polynucleotide to correct the mutant allele and complementary to an anti-sense strand of genomic DNA, wherein the donor polynucleotide comprises a silent mutation in a constant region of the protospacer adjacent motif, e.g., within the final two nucleotides of the protospacer adjacent motif for Streptococcus pyogenes,
producing a biotinylated donor polynucleotide, wherein the donor polynucleotide is biotinylated at the 5′ end or the 3′ end, and
assembling the modified guide RNA, a Cas9 polypeptide, a biotin-binding molecule, and the biotinylated donor polynucleotide.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a is a schematic of the cause of Pompe disease. Pompe disease is caused by two defective copies of acid-α-glucosidase (GAA). This enzyme is responsible for breakdown of glycogen within lysosomes. Without GAA, glycogen build-up can cause downstream health issues. After correction, GAA expresses a functional protein leading to a reduction in lysosomal size.

FIG. 1B is a schematic of editing locations within the GAA locus. Pompe mutants are compound heterozygous at GAA. Allele one contains a point mutation that causes a premature stop codon (GAA:c.[1441=;2237G>A]) while allele two carries a one basepair deletion (GAA:c.[1441delT;2237=]). sgRNAs (underline) were designed to be specific to only the diseased allele by containing mutants (red) within the seed region. ssODNs used for genomic repair contained the wildtype sequence at the mutation site as well as a silent mutation (blue) to remove the PAM site (green) to prevent re-cutting of the corrected allele.

FIG. 1c is a schematic of S1mplex design for repair of compound heterozygous mutations. S1mplexes targeting the 1441delT mutant were labelled with an AlexaFluor®488 compound while S1mplexes targeting the 2237G>A mutation were labelled with an AlexaFluor®647. These RNP species were mixed prior to transfecting into cells and subsequently plated on ArrayEdit platform to conduct high-content analysis.

FIG. 1d shows Sanger sequencing traces of all corrected lines. The unedited line contains mutations at both alleles: 1441delT mutation causes a breakdown of sequence trace, whereas a single point mutation demonstrates a heterozygosity 2237G>A locus. Single corrected clones remain identical to unedited line at unedited locus and contained PAM wobble on the corrected allele. Double corrected line contained PAM wobble at both loci. Wobble A bases in the corrected lines are highlighted to indicate repair from the ssODN. SpCas9 cut site denoted by dotted line, and sequencing chromatograms do not show evidence of undesired NHEJ products.

FIG. 2a, top panel, is a schematic of GAA mRNA used for qRT-PCR. mRNA was analyzed at 3 locations, around the 1441delT locus (solid), the 2237G>A locus (hashed), and at the final 3′ junction (outlined). The middle two panels show an overlay of qRT-PCR and deep sequencing data around each edited locus: analysis around 1441delT loci (solid bars) and analysis around 2237G>A locus (hashed bars). Bars are color coded by sequence identity, either wildtype, mutant, or corrected, from deep sequencing analysis. Bar heights are equivalent to qRT-PCR quantification relative to GAPDH. In all corrected lines, the corrected allele was expressed at a higher frequency than the unedited allele. Double corrected line expressed the highest level of overall mRNA and expressed each allele at approximately equal amounts. The bottom panel shows quantification of total GAA mRNA in unedited, single corrected, and double corrected lines via qRT-PCR. The double corrected line had a significantly higher amount of mRNA than any of the other isolated line (n=3 technical replicates). This is consistent with expression from two active alleles (*p<0.05 ***p<0.005, two-tailed t-test, α=0.05, heteroscedastic). Bar graphs are plotted with standard deviation.

FIG. 2b shows a Western blot for GAA protein. Each of the corrected lines expressed high levels of active protein as well as detectable levels of precursor protein. Unedited cells expressed significantly lower levels of GAA protein but was still above the limit of detection.

FIG. 2c shows GAA activity in cell lysate and cell culture media supernatant as measured by 4-MUG cleavage in acidic conditions. Unedited cells were unable to cleave this substrate, showing there was little to no active protein. All corrected lines had significantly higher activity than unedited cells but were indistinguishable from each other (n=2 technical replicates, *p<0.05, two-tailed t-test, α=0.05, heteroscedastic; Bar graphs plotted with standard deviation).

FIG. 3a is a schematic of enzymatic cross-correction experiments using gene-corrected cardiomyocytes. Unedited iPSC-CMs (red) were supplied media without glucose for 24 hours (orange). After 24 hours media was replaced with media (pink) that had previously been exposed to corrected cell lines (blue) or supplemented with rhGAA. 96 hours after replacement, unedited cells were stained with LysoSensor™ and imaged using confocal microscopy for dye intensity.

FIG. 3b shows quantification of LysoSensor™ intensity in cross-corrected lines 96 hours post media exchange. Each triangle represents a corrected cell identified using CellProfiler™. After 96 hours of daily media changes or supplementing with rhGAA all conditions had significant increase in dye intensity over control conditions. Unedited cells were modified to express histone 2B (H2B)-mCherry to facilitate imaging of the nuclei in these assays. (***p<0.005, n>134, two-tailed t-test, α=0.05, heteroscedastic; bar graphs plotted with standard deviation).

FIG. 3c shows representative images of unedited iPSC-CMs stained with LysoSensor™ in media from unedited and double corrected iPSC-CMs.

FIG. 3d shows representative images of LAMP1 staining in unedited, single corrected, double corrected cells and control PSC-CM and unedited iPSC-CM treated with rhGAA. (scale bars: 10 μm).

FIG. 4a and b show highly precise gene correction using S1mplex strategy in primary fibroblasts from three Pompe disease patients. FIG. 4a shows the percentage of total sequencing reads from S1mplex-edited, primary fibroblasts from Pompe diseased patients. Results indicate gene correction and imprecise editing for 3 different mutations. FIG. 4b shows that percentage of edited alleles that are precisely edited, indicating that S1mplex genome editors can perform as precise-90 editors. Primary fibroblasts were obtained from Coriell Institute. W746X mutation was from Coriell ID: GM04912. D645N mutation was from Coriell ID: GM20090. R660H was from Coriell ID: GM13522.

FIG. 5 shows quantification of LysoSensor™ intensity in each co-culture condition. LysoSensor™ intensity was measured on a per-cell basis using confocal microscopy. Control hPSCs had significantly higher intensity than unedited cells in all conditions. Unedited cells co-cultured with hPSCs also had an increased LysoSensor™ intensity when compared to those cultured alone (*p<0.05, ****p<0.001).

FIG. 6 shows Left: LysoSensor™ quantification per μFeature of two mock transfections after 7 days of growth. hPSCs were significantly more intense than unedited iPSCs on ArrayEdit. Bottom Right: Growth rate of unedited and control hPSCs following a mock transfection to establish a baseline for growth. Growth rates were calculated by measuring the per-day change in the number of cells of the μFeature. Features were graphed as an average of these per day changes. Top Right: LysoSensor™ intensity was plotted against growth rate per μFeature to identify edited colonies. Individual plotted colonies were also assayed for presence of either genome editor (represented in either purple or green), both genome editors (red) and low amounts of genome editors (black). Colonies of interest are identified as those with high genome editor expression and lower growth rates, presumably arising from the stress of genome editing. Dashed lines indicate regions of interest. Also included is a magnification of quadrant II from panel. μFeatures in this region were selected for genomic analysis to isolate edited clones.

FIG. 7 shows karyotypes of all isolated gene-corrected lines as well as unedited cells. No abnormalities were detected at a band resolution of 500.

FIG. 8 shows a schematic for long PCR covering both Cas9 cut sites. Primers are denoted by arrows. The expected PCR amplicon is 7959 bp in length.

FIG. 9 shows a gel analysis of long range PCR described in panel c and FIG. 2a in each isolated cell line. No significant deviances from the expected length were detected, and no other significant bands were observed. WA09 control cells are hPSCs.

FIG. 10 shows Sanger sequencing traces of long range PCR shown in panel FIG. 9. SNPs were observed showing that PCR products were a result of amplification from both alleles within the cells.

The above-described and other features will be appreciated and understood by those skilled in the art from the following detailed description, drawings, and appended claims.

### DETAILED DESCRIPTION

For individuals with an autosomal recessive disease (e.g., cystic fibrosis, sickle cell anemia, and Tay-Sachs disease), the mutated gene is located on one of the nonsex chromosomes (autosomes), and both alleles of the gene carry mutations. The parents of an individual with symptoms of an autosomal recessive disease generally each carry one copy of the mutated gene, but they do not show obvious symptoms of the disease. Therefore, it has been assumed that correcting only one allele of the mutated gene would be sufficient to rescue the disease, and that additional genomic surgery to repair the second allele may subject a patient to undue risk. The inventors have tested this assumption with CRISPR-Cas9 gene editing to systematically correct both mutated alleles within the same cell from an autosomal recessive, infantile-onset case of Pompe disease. Unexpectedly, the inventors have shown that a CRISPR-Cas9 gene editing system can correct both mutated alleles.

The inventors previously developed a S1mplex strategy for modified guide RNAs such as sgRNAs and their RNP complexes with Cas9. The S1mplex tool exploits high affinity interactions between a short RNA aptamer and streptavidin to promote more faithful writing of the human genome. Slmplex modified guide RNAs or traditional guide RNAs can be used in the methods described herein.

In an aspect, these RNP-containing complexes can be assembled outside the cell to a desired stoichiometry and delivered as an all-in-one gene-editing nanoparticle together with a donor nucleic acid template. In addition, the complexes can be easily decorated with additional moieties such as fluorophores or Qdots to enrich for edited cells. Use of these particles with a biotinylated ssODN reduced heterogeneity in delivery among RNPs and nucleic acids within human cells and enriches the ratio of precisely-edited to imprecisely-alleles edited alleles up to 18-fold higher than standard RNP methods, approaching a ratio of four precise edits to every one imprecise edit. Further functionalization with a unique fluorophore enables multiplexed editing and enrichment of precisely edited populations through cell sorting. Taken together, advances with the S1mplex tool generates chemically-defined reagents to promote precise editing of the human genome.

The S1mplex strategy is inspired by CRISPR display that leverages structural studies of the RNP to identify locations in the sgRNA sequence where RNA aptamers could be tolerated.

CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA interacts with Cas9 and directs the nuclease activity of Cas9 to target DNA sequences complementary to those present in the guide RNA. Guide RNA base pairs with complementary sequences in target DNA. Cas9 nuclease activity then generates a double-stranded break in the target DNA.

CRISPR/Cas9 is an RNP complex. CRISPR RNA (crRNA) includes a 20 base protospacer element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

sgRNA refers to a single RNA species which combines the tracrRNA and the crRNA and is capable of directing Cas9-mediated cleavage of target DNA. An sgRNA thus contains the sequences necessary for Cas9 binding and nuclease activity and a target sequence complementary to a target DNA of interest (protospacer sequence). In general, in an sgRNA, the tracrRNA and the crRNA are connected by a linker loop sequence. sgRNAs are well-known in the art. While sgRNA is generally used throughout this disclosure, two-part guide RNAs containing a crRNA and a tracrRNA can also be employed.

As used herein, a guide RNA protospacer sequence refers to the nucleotide sequence of a guide RNA that binds to a target DNA sequence and directs Cas9 nuclease activity to the target DNA locus. In some embodiments, the guide RNA protospacer sequence is complementary to the target DNA sequence. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the targeted sequence includes a protospacer adjacent motif (PAM) adjacent to the protospacer region which is a sequence recognized by the CRISPR RNP as a cutting site. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyo genes has a NGG trinucleotide PAM motif; the PAM motif of N. meningitidis Cas9 is NNNNGATT; the PAM motif of S. thermophilus Cas9 is NNAGAAW; and the PAM motif of T. denticola Cas9 is NAAAAC.

A modified guide RNA is a one-part or two-part RNA capable of directing Cas-9-mediated cleavage of target DNA. A modified sg RNA is a single RNA species capable of directing Cas9-mediated cleavage of target DNA. A modified sgRNA, for example, comprises sequences that provide Cas9 nuclease activity, a protospacer sequence complementary to a target DNA of interest, and an aptamer that binds a biotin-binding molecule. The linker loop that connects the tracrRNA and the crRNA in an sgRNA can be replaced with an aptamer that binds a biotin-binding molecule such as a streptavidin-binding aptamer. Unexpectedly, the modified sgRNAs can bind both Cas9 protein and streptavidin and form active RNP complexes which induce error-prone DNA repair less frequently than standard CRISPR-Cas9 RNP complexes.

In an aspect, a modified guide RNA, comprises
a crRNA comprising, a single-stranded protospacer sequence and a first complementary strand of a binding region for the Cas9 polypeptide, and
a tracrRNA comprising a second complementary strand of the binding region for the Cas9 polypeptide,
wherein the crRNA or the tracrRNA comprises an aptamer that binds a biotin-binding molecule,
wherein the crRNA and the tracrRNA hybridize through the first and second complementary strands of the binding region for the Cas9 polypeptide,
wherein the single stranded protospacer region comprises

(SEQ ID NO: 1)
CTCGTTGTCCAGGTAGGCCC,
(SEQ ID NO: 60)
GGACCACCAGCTCCTGTAGG,
(SEQ ID NO: 61)
GCCCAGGAAGCCGCAGACGT,
or
(SEQ ID NO: 62)
CAGAGGAGCTGTGTGTGCAC
(SEQ ID NO: 2)
TGGACCACCAGCTCCTGTAG.

In another aspect, the crRNA and the tracrRNA form an sgRNA, the sgRNA comprising from 5′ to 3′,
the single-stranded protospacer sequence,
the first complementary strand of a binding region for the Cas9 polypeptide,
the aptamer that binds a biotin-binding molecule, and
the second complementary strand of the binding region for the Cas9 polypeptide.

More specifically, a modified sgRNA comprises, from 5′ to 3′, a single-stranded protospacer sequence, a first complementary strand of a binding region for the Cas9 polypeptide, an aptamer that binds a biotin-binding molecule, and a second complementary strand of the binding region of the Cas9 protein. In an embodiment, in the secondary structure of the modified sgRNA, the stem forms a stem-loop structure with the aptamer that binds the biotin-binding molecule.

In another aspect,
a crRNA comprises a single-stranded protospacer sequence and a first complementary strand of a binding region for the Cas9 polypeptide, and
a tracrRNA comprising a second complementary strand of the binding region for the Cas9 polypeptide,
wherein the crRNA and the tracrRNA hybridize through the first and second complementary strands of the binding region for the Cas9 polypeptide,
wherein the single stranded protospacer region comprises

(SEQ ID NO: 1)
CTCGTTGTCCAGGTAGGCCC,
(SEQ ID NO: 2)
TGGACCACCAGCTCCTGTAG,
(SEQ ID NO: 60)
GGACCACCAGCTCCTGTAGG,
(SEQ ID NO: 61)
GCCCAGGAAGCCGCAGACGT,
or
(SEQ ID NO: 62)
CAGAGGAGCTGTGTGTGCAC.

The single-stranded protospacer region can comprise 17 to 20 nucleotides. Exemplary binding regions for Cas9 polypeptides comprise 10 to 35 base pairs.

In an aspect, the aptamer that binds a biotin-binding molecule forms a stem-loop structure. The stem portion of the stem-loop structure optionally forms a contiguous double strand with the double-stranded binding region for the Cas9 polypeptide. The stem portion of the aptamer can comprise 9 to 15 base pairs, while the loop comprises 30 nucleotides. The aptamer may contain more than one stem-loop structure. The length of the stem portion of the aptamer is not critical and can be adjusted depending on the application of the modified guide RNA.

A “Cas9” polypeptide is a polypeptide that functions as a nuclease when complexed to a guide RNA, e.g., an sgRNA or modified sgRNA. The Cas9 (CRISPR-associated 9, also known as Csn1) family of polypeptides, for example, when bound to a crRNA:tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case1, Cse 2, Csy 1, Csy 2, Csy 3, GSU0054, Cas 10, Csm 2, Cmr 5, Cas10, Csx11, Csx10, and Csf 1. Additional Class 2 Cas9 polypeptides include Csn 2, Cas4, C2c1, C2c3 and Cas13a.

Exemplary Cas9 polypeptides include Cas9 polypeptide derived from Streptococcus pyogenes, e.g., a polypeptide having the sequence of the Swiss-Prot accession Q99ZW2 (SEQ ID NO: 3); Cas9 polypeptide derived from Streptococcus thermophilus, e.g., a polypeptide having the sequence of the Swiss-Prot accession G3ECR1 (SEQ ID NO: 4); a Cas9 polypeptide derived from a bacterial species within the genus Streptococcus; a Cas9 polypeptide derived from a bacterial species in the genus Neisseria (e.g., GenBank accession number YP_003082577; WP_015815286.1 (SEQ ID NO: 5)); a Cas9 polypeptide derived from a bacterial species within the genus Treponema (e.g., GenBank accession number EMB41078 (SEQ ID NO: 6)); and a polypeptide with Cas9 activity derived from a bacterial or archaeal species. Methods of identifying a Cas9 protein are known in the art. For example, a putative Cas9 protein may be complexed with crRNA and tracrRNA or sgRNA and incubated with DNA bearing a target DNA sequence and a PAM motif.

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

In addition, to the modified sgRNA and the Cas9 polypeptide or active fragment thereof, an RNP complex may further comprise a biotin-binding molecule such as an avidin such as avidin, streptavidin, or neutravidin which bind with high affinity to the aptamer that binds the biotin-binding molecule in the modified sgRNA. Avidin, streptavidin and neutravidin are tetramers, and each subunit can bind biotin with equal affinity. Avidin, streptavidin and neutravidin variants that contain one, two or three biotin binding sites are also available and may be employed in the complex.

When the RNP complex comprises a biotin-binding molecule, the complex can further comprise a biotinylated molecule which associates with the complex via the biotin-binding molecule. The biotinylated molecule can target the RNP complex to a specific cell type, organ or tissue. For example, PEG-coated gold nanoparticles exhibit size-dependent in vivo toxicity; the renal clearance of quantum dots can be controlled; and the accumulation of PEGylated silane-coated magnetic iron oxide nanoparticles has been shown to be size dependent.

In one embodiment, the biotinylated molecule is a biotinylated oligodeoxynucleotide, such as a biotinylated donor DNA template. Homologous recombination can insert an exogenous polynucleotide sequence into the target nucleic acid cleavage site. An exogenous polynucleotide sequence can be called a donor polynucleotide or a donor sequence. In some embodiments, a donor polynucleotide, a portion of a donor polynucleotide, a copy of a donor polynucleotide, or a portion of a copy of a donor polynucleotide can be inserted into a target nucleic acid cleavage site. A donor polynucleotide can be single-stranded DNA, double-stranded DNA, RNA, or a duplex of RNA and DNA. A donor polynucleotide can be a sequence that does not naturally occur at a target nucleic acid cleavage site. In some embodiments, modifications of a target nucleic acid due to NHEJ and/or HDR can lead to, for example, mutations, deletions, alterations, integrations, gene correction, gene replacement, transgene insertion, nucleotide deletion, gene disruption, and/or gene mutation. The process of integrating non-native nucleic acid(s) into genomic DNA can be referred to as “genome engineering”.

In an embodiment, the biotinylated molecule is a nanoparticle, such as a quantum dot, a gold particle, a magnetic particle, a polymeric nanoparticle. In another embodiment, the biotinylated molecule is a biotinylated fluorescent dye such as Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexafluor® 488 biocytin, Alexafluor® 546, Alexa fluor® 549, lucifer yellow cadaverine biotin-X, Lucifer yellow biocytin, Oregon green 488 biocytin, biotin-rhodamine and tetramethylrhodamine biocytin. Biotinylated molecule may also be a peptide, proteins or protein domains, specifically antibodies and Fab domains.

In another embodiment, the biotinylated donor polynucleotide comprises a contrast agent, a cell targeting ligand, a tissue targeting ligand, or a peptide.

In another aspect, the biotin-binding molecule can be covalently linked to a donor polynucleotide, a nanoparticle, or a dye molecule either directly or via a linker molecule, using, for example a disulfide linker. The bound biotin-binding molecule can then bind the aptamer of the modified sgRNA. Additional biotinylated donor polynucleotides, nanoparticle, contrast agent, or dye molecules can then be associated with the bound biotin-binding molecule. Alternatively, the biotin-binding molecule can be associated with the biotinylated molecule prior to adding to modified sgRNA.

A method of modifying a target gene in a patient or in a patient-derived cell, wherein the patient has an autosomal recessive disorder with compound heterozygous mutations comprises
delivering to the patient or cell a first modified guide RNA, a second modified guide RNA, a Cas9 polypeptide, a biotin-binding molecule, a first biotinylated donor polynucleotide, and a second biotinylated donor polynucleotide, wherein each modified guide RNA comprises,

a crRNA comprising, a single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,

wherein the single-stranded protospacer sequence of the modified guide RNA hybridizes to a sequence in the target gene to be modified.
wherein the first modified guide RNA and the first biotinylated donor polynucleotide correct a first diseased allele,
wherein the second modified guide RNA and the second biotinylated donor polynucleotide correct a second diseased allele.

Exemplary patient-derived cells comprises an induced pluripotent stem cell, a progenitor cell, a mesenchymal stem cell, or a tissue-specific stem cell. Exemplary tissue-specific stem cells comprises a skeletal stem cell, a hematopoietic stem cell, an epithelial stem cell, or a neural stem cell.

In an embodiment, a first RNP complex comprises the first modified guide RNA, the Cas9 polypeptide, the biotin-binding molecule and the first biotinylated donor polynucleotide; and a second RNP complex comprises the second modified guide RNA, the Cas9 polypeptide, the biotin-binding molecule and the second biotinylated donor polynucleotide.

In another embodiment, the first modified guide RNA, the second modified guide RNA and the Cas9 polypeptide are expressed from one or more viral vectors. For example, a first viral vector expresses the first modified guide RNA, and a second viral vector expresses the second modified guide RNA, and a third viral vector expresses the Cas9 polypeptide.

Exemplary patients are human patients. Exemplary human autosomal recessive disorders are aspartylglucosaminuria, Batten disease, cystinosis, Fabry disease, Gaucher Disease Type I, II or III, Pompe Disease, Tay Sachs Disease, Sandhoff Disease, Metachromatic leukodystrophy, Mucolipidosis Type, I, II/III or IV, Hurler Disease, Hunter disease, Sanfilippo disease Types A,B,C,D, Morquio disease Types A and B, Maroteaux-Lamy disease, Sly disease, Niemann-Pick Disease Types A/B, C1 or C2, or Schindler Disease Types I or II.

In an embodiment, the autosomal recessive disorder is a human lysosomal storage disorder, such as Pompe disease.

A method of treating a patient with an autosomal recessive disorder with compound heterozygous mutations comprises transplanting the cell made by the methods described herein into the subject. Lysosomal storage diseases are caused by an inborn error of metabolism that results in the absence or deficiency of an enzyme, leading to the inappropriate storage of material in various cells of the body. Most lysosomal storage disorders are inherited in an autosomal recessive manner.

In an embodiment, modifying the target gene increases or decreases the expression of a gene product of the target gene.

In another embodiment, modifying the target gene comprises high-fidelity homology-directed repair (HDR).

In another embodiment, modifying the target gene comprises the addition of a genetic functionality, or the correction of a mutation.

In yet another embodiment, modifying the target gene creates a double strand break (DSB) which is repaired by a non-homologous end joining (NHEJ) cell repair mechanism generating indels thereby modifying the polynucleotide sequence of the target gene.

In a further embodiment, modifying the target gene creates a DSB which is repaired by a homologous recombination (HDR) cell repair mechanism incorporating a donor DNA sequence thereby modifying the polynucleotide sequence of the target gene.

In another aspect, the S1m-sgRNAs described herein can be used for the excision of genomic DNA. In an aspect, two S1m-sgRNAs can be employed simultaneously, wherein each S1m-sgRNA targets an end of the region to be excised. As shown in Example 12, human cells contain the properly excised region of genomic DNA.

Delivery of polynucleotides and RNPs of the present disclosure to cells, in vitro, or in vivo, may be achieved by a number of methods known to one of skill in the art. These methods include lipofection, electroporation, nucleofection, microinjection, biolistics, liposomes, immunoliposomes, polycation or lipid:nucleic acid conjugates. Lipofection is well known and lipofection reagents are sold commercially. Cationic and neutral lipids that are suitable for efficient receptor-recognition lipofection of polynucleotides are described in the art.

Lipid:nucleic acid complexes, including targeted liposomes such as immunolipid complexes, and the preparation of such complexes is well known to one of skill in the art.

Electroporation can be used to deliver the polynucleotides and RNPs of the present disclosure. In these methods, the polynucleotides or RNPs are mixed in an electroporation buffer with the target cells to form a suspension. This suspension is then subjected to an electrical pulse at an optimized voltage, which creates temporary pores in the phospholipid bilayer of the cell membrane, permitting charged molecules like DNA and proteins to be driven through the pores and into the cell. Reagents and equipment to perform electroporation are sold commercially.

Biolistic, or microprojectile delivery, can be used to deliver the polynucleotides and RNPs of the present disclosure. In these methods, microprojectiles, such as gold or tungsten, are coated with the polynucleotide by precipitation with calcium chloride, spermidine or polyethylene glycol. The microprojectile particles are accelerated at high speed into a cell using a device such as the BIOLISTIC® PDS-1000/He Particle Delivery System (Bio-Rad; Hercules, Calif.).

In another embodiment, a viral vector expressing the modified guide RNA of the present disclosure, a viral vector expressing a Cas9 polypeptide and biotinylated donor DNA template (e.g., a biotinylated donor DNA template), can be transfected into a cell, such as a human cell. Human cells include human pluripotent stem cell lines and primary blood cell such as hematopoietic stem and progenitor cells and T-cells. Once editing has occurred in the cell line, the cells can be differentiated and transplanted into a subject or used for drug development.

In some embodiments, the polynucleotides of the present disclosure may also comprise modifications that, for example, increase stability of the polynucleotide. Such modifications may include phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and amino alkylphosphoramidates, phosphorodiamidates, thionophosphoramidates, thionoalkylphosphonates, thionoalkylphosphotriesters, selenophosphates, and boranophosphates having normal 3′-5′ linkages, 2-5′ linked analogs, and those having inverted polarity wherein one or more internucleotide linkages is a 3′ to 3′, a 5′ to 5′ or a 2′ to 2′ linkage. Exemplary nucleic acid-targeting polynucleotides having inverted polarity can comprise a single 3′ to 3′ linkage at the 3′-most internucleotide linkage (i.e., a single inverted nucleoside residue in which the nucleobase is missing or has a hydroxyl group in place thereof). Various salts (e.g., potassium chloride or sodium chloride), mixed salts, and free acid forms can also be included.

In some embodiments, the polynucleotides of the present disclosure may also contain other nucleic acids, or nucleic acid analogues. An example of a nucleic acid analogue is peptide nucleic acid (PNA).

In an embodiment, a method of modifying a target gene in a patient or in a patient-derived cell, wherein the patient has Pompe disease, comprises
delivering to the patient or cell a first modified guide RNA, a second modified guide RNA, a Cas9 polypeptide, a biotin-binding molecule, a first biotinylated donor polynucleotide, and a second biotinylated donor polynucleotide, wherein each modified guide RNA comprises,

a crRNA comprising, a single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,

wherein the single-stranded protospacer sequence of the modified guide RNA hybridizes to a sequence in the target gene to be modified.
wherein the first modified guide RNA and the first biotinylated donor polynucleotide correct a first diseased allele,
wherein the second modified guide RNA and the second biotinylated donor polynucleotide correct a second diseased allele,
wherein the single stranded protospacer region of the first modified guide RNA comprises CTCGTTGTCCAGGTAGGCCC (SEQ ID NO: 1) and the single stranded protospacer region of the second guide RNA comprises TGGACCACCAGCTCCTGTA (SEQ ID NO: 2).

In another embodiment, a method of modifying a target gene in a patient or in a patient-derived cell, wherein the patient has Pompe disease, comprises
delivering to the patient or cell a first guide RNA, a second guide RNA, a Cas9 polypeptide, a first donor polynucleotide, and a second donor polynucleotide, wherein each guide RNA comprises,

a crRNA comprising, a single-stranded protospacer sequence and a first complementary strand of a binding region for the Cas9 polypeptide, and

a tracrRNA comprising a second complementary strand of the binding region for the Cas9 polypeptide,

wherein the crRNA and the tracrRNA hybridize through the first and second complementary strands of the binding region for the Cas9 polypeptide,

wherein the single-stranded protospacer sequence of the modified guide RNA hybridizes to a sequence in the target gene to be modified.
wherein the first guide RNA and the first donor polynucleotide correct a first diseased allele,
wherein the second guide RNA and the second donor polynucleotide correct a second diseased allele,
wherein the single stranded protospacer region of the first modified guide RNA comprises CTCGTTGTCCAGGTAGGCCC (SEQ ID NO: 1) and the single stranded protospacer region of the second guide RNA comprises TGGACCACCAGCTCCTGTA (SEQ ID NO: 2).

Also included herein are methods of making RNP complexes, specifically first and second RNP complexes that provide biallelic correction. In an embodiment, a method of making an RNP complex, comprises
selecting a single stranded protospacer sequence by identifying a mutant allele to be corrected, wherein the mutant allele is within 0 to 100 bases of a protospacer adjacent motif,
producing a modified guide RNA comprising the selected single stranded protospacer region, wherein the modified guide RNA comprises

a crRNA comprising the single-stranded protospacer sequence and a first complementary strand of a binding region for the Cas9 polypeptide, and

a tracrRNA comprising a second complementary strand of the binding region for the Cas9 polypeptide,

wherein the crRNA or the tracrRNA comprises an aptamer that binds a biotin-binding molecule,

wherein the crRNA and the tracrRNA hybridize through the first and second complementary strands of the binding region for the Cas9 polypeptide,
selecting a donor polynucleotide to correct the mutant allele and complementary to an anti-sense strand of genomic DNA, wherein the donor polynucleotide comprises a silent mutation in a constant region of the protospacer adjacent motif, e.g., within the final two nucleotides of the protospacer adjacent motif for Streptococcus pyogenes,
producing a biotinylated donor polynucleotide, wherein the donor polynucleotide is biotinylated at the 5′ end or the 3′ end, and
assembling the modified guide RNA, a Cas9 polypeptide, a biotin-binding molecule, and the biotinylated donor polynucleotide.

In an embodiment, the donor polynucleotide is asymmetric around a cut site. In another embodiment, the method further comprises repeating the method and producing a second RNP complex, wherein the second RNP complex corrects a second mutant allele to result in a biallelic correction.

The invention is further illustrated by the following non-limiting examples.

### EXAMPLES

### Methods

Cell Culture: All hPSCs were maintained in mTeSR1™ medium on Matrigel® (WiCell) coated tissue culture polystyrene plates (BD Falcon). Cells were passaged every 4-5 days at a ratio of 1:8 using Versene™ solution (Life Technologies). Patient derived human induced pluripotent stem cell line Pompe GM04192 was a gift from the T. Kamp and M. Suzuki (UW-Madison) labs. Cardiomyocytes derived from hPSC and iPSC cultures were maintained in RPMI/B27 on Matrigel® (WiCell) coated polystyrene plates (BD Falcon). Patient derived fibroblast lines were obtained from Coriell Institute with different GAA mutations and cultured in DMEM supplemented with 10% FBS and 1% Penicillin/Streptomycin. All cells were maintained at 37° C. in 5% CO₂ and tested monthly for possible mycoplasma contamination.

Cardiomyocyte Differentiation: hPSCs and iPSCs were differentiated into cardiomyocytes using a small molecule-directed differentiation protocol in a 12-well plate format as previously described. Briefly, all adherent hPSCs and iPSCs were dissociated in TrypLE™ solution (Life Technologies), counted with a hemocytometer, and centrifuged at 200× g for 5 minutes. Cells were plated at a density between 0.5-1×10⁶ cells/well depending on cell line. Once tissue culture plate wells reached 100% confluency (day 0), medium in each well was replaced with a solution containing ml RPMI/B27-Insulin (Life Technologies), 12 μM CHIR99021 (BioGems 25917), and 1 μg/ml Insulin solution (Sigma-Aldrich 19278). Exactly 24 hours later (day 1) medium in each well was removed and replaced with RPMI/B27-insulin. Exactly 48 hours after (day 3) half of the spent medium was collected. To this, an equal volume of fresh RPMI/B27-Insulin was mixed. This combined media was then supplemented with 7.5 μM IWP2 (BioGems 75844). Two days later (day 5) medium in each well was replaced with RPMI/B27-Insulin. Two days (day 7) later and every three days following, spent medium was replaced with RPMI/B27. Spontaneous contraction was generally observed between days 12-16 of differentiation.

Creation of Slrn-sgRNAs: S1m-sgRNAs were synthesized as previously described. S1m gBlocks were annealed with Phusion™ polymerase (New England Biolabs) under the following thermocycler conditions: 98° C. for 30 sec followed by 30 cycles at 98° C. for 10 s, and 72° C. for 15 s with a final extension at 72° C. for 10 minutes. S1m cDNA was annealed with Phusion™ polymerase (New England Biolabs) under the following thermocycler conditions: 98° C. for 30 sec followed by 30 cycles at 98° C. for 10 s, 60° C. for 10 s, and 72° C. for 15 s with a final extension at 72° C. for 10 minutes. In vitro transcription was performed with the MEGAShortscript™ T7 Kit (Thermo Fisher Scientific) according to manufacturer's instructions.

For guides for fibroblast transfection, in vitro transcription was performed using HiScribe™ T7 RNA synthesis Kit (New England Biolabs).

Genome Editor Deliver: All hPSC transfections were performed using the 4D-Nucleofector System™ (Lonza) in P3 solution using protocol CA-137. 50 pmol Cas9, 60 pmol sgRNA, 50 pmol streptavidin, and 60 pmol ssODN were used to form particles per ssODN-S1mplex as described above. Cells were then harvested using TrypLE™ (Life Technologies) and counted. 2×10⁵ cells per transfection were then centrifuged at 100× g for 3 minutes. Excess media was aspirated, and cells were resuspended using 20 μL of RNP solution per condition. After nucleofection, samples were incubated in nucleocuvettes at room temperature for 15 minutes prior to plating into 2*10⁴ cells per well on ArrayEdit in mTeSR™ media+10 μM ROCK inhibitor. Media was changed 24 hours post transfection and replaced with mTeSR™ medium. Fibroblast transfections were performed in 24 well plates using 50,000 cells/well using 2 μl Lipofectamine® 2000/well (0.5 μg Cas9/well and sgRNA, streptavidin and ssODN at a 1:1:1:1 molar ratio).

Synthesis of ArrayEdit Platform: μCP was performed using previously described methods. The surface modification involved printing of an alkanethiol initiator to nucleate the polymerization of hydrophilic poly(ethylene glycol) (PEG) chains. Briefly, double sided-adhesive was attached to the bottom of a standard tissue culture plate, after which a laser cutter was used to cut out the well bottoms. Using previously described chemistry, patterns were transferred to gold-coated glass via a polydimethylsiloxane stamp after which the glass was submerged in a poly(ethylene glycol) (PEG) solution overnight to build hydrophilic PEG chains surrounding μFeatures. Standard tissue culture plates with well bottoms cut out were then fastened to processed sheets using a custom-made alignment device.

High-Content Analysis: Automated microscopy was performed using a Nikon Eclipse TI epifluorescent scope. A 20×20 grid with one μFeature per image was established and maintained so that each feature imaged was consistent each day. Nikon Perfect Focus was used to ensure that all colonies were in the same Z-plane and LysoSensor™ intensity was measured accurately. Images were processed using CellProfiler™ to count the number of nuclei and quantify LysoSensor™ intensity.

Genomic Sequencing: DNA was isolated from cells using DNA QuickExtract™ (Epicentre) following TrypLE™ treatment and centrifugation. Extracted DNA was incubated at 65° C. for 15 min, 68° C. for 15 min, and 98° C. for 10 min. Genomic PCR was performed using AccuPrime™ HiFi Taq (Life Technologies) and 500 ng of genomic DNA according to manufacturer's instructions. Long (8kb) PCR reactions were thermocycled using an extension step of 10 minutes. All genomic PCR products were then submitted to the University of Wisconsin-Madison Biotechnology Center for DNA sequencing.

RT- and qPCR: RNA was isolated from cells using RNA QuickExtract™ (Epicentre) following the manufacturer's protocol. 100 ng of extracted RNA was reverse transcribed using Superscript® IV Reverse Transcriptase (Invitrogen). Endpoint PCR amplification of the cDNA product was performed following the manufacturer's instructions using AccuPrime™ HiFi Taq (Life Technologies) and 1 μl of cDNA Product. Efficacy of the endpoint PCR was performed via gel electrophoresis of the PCR product in a 1% agarose gel.

The qPCR reaction was set up in triplicate for each cell line and sequence (GAPDH, dT, 746, and GAA), by mixing 10 μl iTaq™ Universal SYBR® Green Supermix (Bio-Rad), 0.5 μl sequence specific forward primer, 0.5 μl sequence specific reverse primer, 1 μl cDNA product, and 8 μl water. qPCR analysis was performed in a CFX96 Real Time PCR System under the following thermocycling conditions: 95° C. for 30 s followed by 35 cycles of 95° C. for 5 s, and 60° C. for 30 s, with a melt curve analysis increasing stepwise from 65° C. to 95° C. in increments of 0.5° C.

Next Generation Sequencing Analysis: A custom python script was developed to perform sequence analysis. For each sample, sequences with frequency of less than 1000 were filtered from the data. Sequences in which the reads matched with primer and reverse complement subsequences classified as “target sequences”. Target sequences were aligned with corresponding wildtype sequence using global pairwise sequence alignment. The frequency, length, and position of matches, insertions, deletions, and mismatches were all tracked in the resulting aligned sequences.

Western Blotting: Protein expression of GAA and β-Actin was determined in each cell line. Following cell lysis in ice-cold RIPA buffer supplemented with protease and phosphatase inhibitors and EDTA (5 mM), protein concentration was determined (DC Protein Assay, BioRad). Forty μg of protein from each cell line was loaded into a 4-12% Bis-Tris precast gel (Criterion XT, BioRad) and gel electrophoresis performed. Proteins were then transferred to a nitrocellulose membrane and blocked in filtered 5% nonfat dry milk in TB S-T (Tris buffered saline, 0.15% Tween20) for 1 hour at room temperature. The membrane was then incubated overnight at 4° C. with GAA (Abcam ab137068, 1:1000) and β-Actin (Millipore, MAB1501, 1:40,000) primary antibodies. Following the incubation period, the membrane was washed in TBS-T and incubated with appropriate horseradish peroxidase secondary antibodies (Goat Anti-Rabbit IgG, Abcam ab205718, 1:2000; Anti-Mouse IgG, Cell Signaling Technologies 7076 1: 20,000) for 1 hour. The membrane was washed again in TBS-T, and then developed (SuperSignal™ West Pico Plus Chemiluminescent Substrate, Thermo Scientific) for 5 minutes using a ChemiDoc-It™ 2 Imaging System (UVP) and imaged.

GAA Activity Assay: Acid glucosidase activity was measured by hydrolysis of 4-methylumbelliferyl-D-glucoside (4-MUG, Sigma M-9766) at pH 4 to release the fluorophore 4-methylumbelliferone (4-MU) as previously described. Briefly, 4-MUG was incubated with 10 μL protein lystate in 0.2M sodium acetate for one hour at 37° C. Fluoresence was then measured using a Glomax® plate reader (Progmega) and activity was calculated using a standard curve.

Immunocytochemistry: Live cell imaging of lysosome intensity was done using LysoSensor™ Green (Life Technologies L7535). Dye was mixed in culture media at a 1:1000 dilution prior to adding media to wells. Cells were then incubated for 5 minutes in LysoSensor™ solution. Media was then aspirated, and cells were washed 2× with PBS. All imaging was done within one hour of staining.

To assay for pluripotency markers, hPSC cultures were fixed using 4% PFA and incubated at room temperature for 10 minutes. Cells were then permeabilized using 0.05% Triton™ X-100 and incubated for 10 minutes. Following two washes with 5% goat serum, NANOG antibody (R&D Systems AF1997, 1: 200) and TRA-1-60 antibody (Millipore MAB5360, 1: 150), was added to cells and incubated overnight at 4° C. The next day, cells were rinsed twice with 5% goat serum and then incubated with a donkey anti-goat secondary antibody (Life Technologies A11055 1: 500) for one hour at room temperature. Cells were then washed twice with PBS and mounted for imaging.

Cardiomyocyte cultures were processed in the same manner as above. After permeabilization, cells were incubated with anti-sarcomeric alpha-actinin (Abcam ab68167 1: 250) overnight at 4° C. The next day, cells were rinsed twice with 5% goat serum and then incubated with a goat anti-rabbit secondary antibody (Santa Cruz Biotech sc-362262, 1: 500.

Media Exchange: Cardiomyocytes were cultured in RPMI/B27+ insulin and media was exchanged every two days. As a normal media exchange, diseased and corrected cells were introduced to RPMI/B27+insulin/−glucose. 24 hours post change, cells were stained with LysoSensor™ as described above to determine a baseline fluorescent intensity. After staining, media was replaced with media from either corrected or healthy lines and cultured for an additional 24 hours. After incubation, cells were again stained with LysoSensor™ and imaged using confocal microscopy.

Isolation of corrected iPSCs: On day one post plating, we measured the presence of S1mplex within the nucleus as well as identified μFeatures that contained only one cell to ensure clonal populations. On days two through six, we measured the number of cells to obtain a growth rate via day over day change. Finally, on day 7, we measured the number of cells as well as stained cells with LysoSensor™ to identify populations that may have been edited using phenotypic recovery as a marker. We also mock transfected WA09 and Pompe iPSCs and plated them on ArrayEdit and subjected them to the same high content analysis as a control. After 7 days we plotted each individual μFeature by its LysoSensor™ intensity and growth rate and color coded each feature by the presence of S1mplexes on day 1. We observed a large population of clones that grew slower than the slowest mock transfected Pompe colonies suggesting that that population may undergo editing events. By comparing LysoSensor™ intensity we also observed that many of the μFeatures within the wells had higher, and therefore more acidic organelles than mock transfected Pompe iPSCs. In fact, many of the μFeatures had similar intensities to control WA09 colonies. By combining these data with the presence of S1lmplexes we were able to select colonies that were potentially preferably edited at either locus individually or both simultaneously. Using this knowledge, we selected colonies of interest for expansion and analysis.

Following expansion of selected clones, we analyzed each one at both loci for the correction of mutations. We also designed the introduction of a PAM codon wobble to ensure that supplied donor DNA was the source for repair. When we looked at colonies that only had the presence of one S1mplex on day 1 we obtained clones that were edited at the specified allele. Interestingly we did not isolate any clones that had indel mutations caused by NHEJ. Further, colonies that were positive for one S1mplex were not observed to be edited at the other locus. We next analyzed clones positive for both S1mplexes and managed to obtain a clone that was edited at both alleles and also contained the PAM wobble. There was also one colony that contained one PAM mutation while the other allele was repaired but did not introduce the novel mutation. Importantly, across all screened clones we did not obtain any that contained indel products. We then selected one clone from each population (edited at either allele individually, or both) to assay for phenotypic recovery.

### Example 1: Correction of Two Diseased Alleles Within Pompe iPSCs

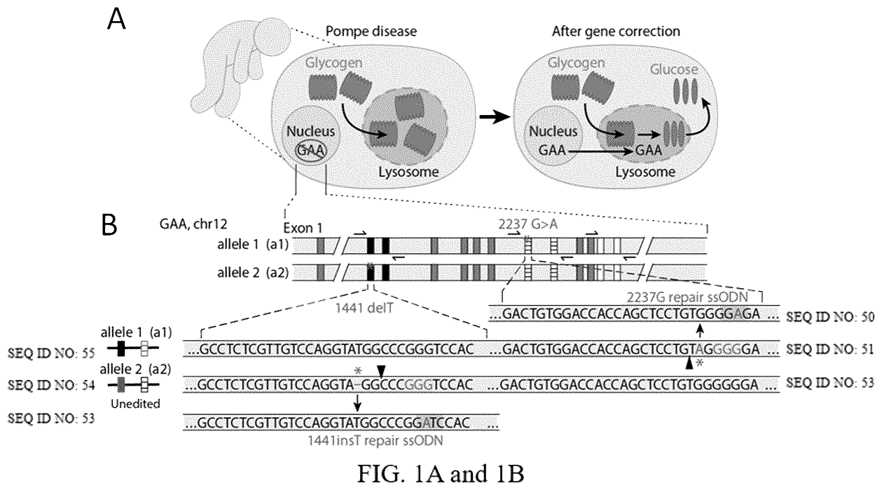

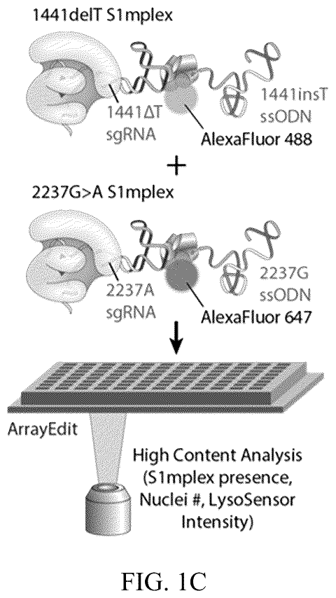

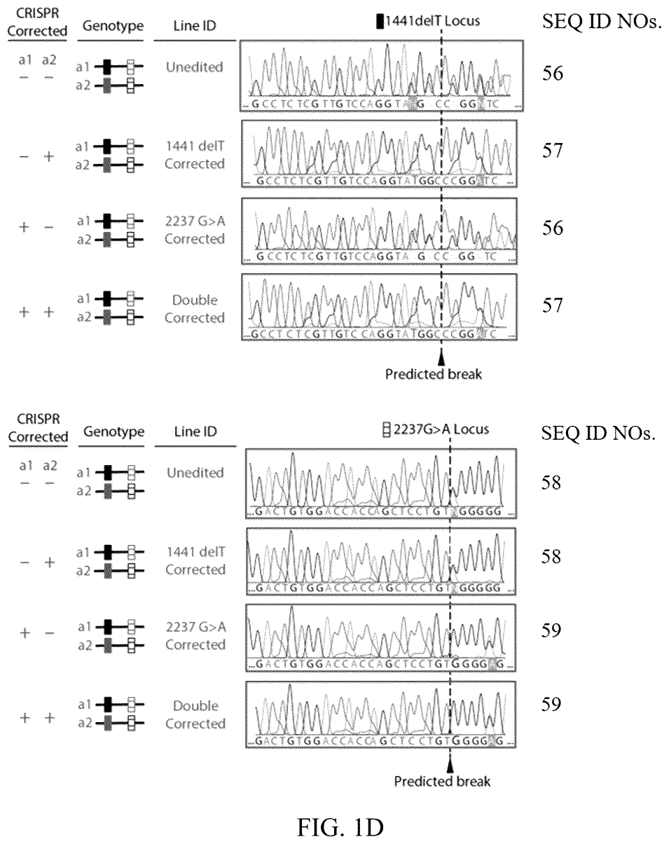

To explore whether two corrected endogenous alleles within the same cell is possible, several clonal isogenic iPSC lines were generated by CRISPR-Cas9 gene editing of an iPSC line derived from a patient with infantile-onset Pompe disease. In this line, compound heterozygous GAA mutations responsible for the disease phenotype are a deletion of a thymidine nucleotide at position 1441 (GAA:c.[1441delT], “1441delT”) causing a frameshift, and premature stop codon on one allele, and a G>A conversion at nucleotide 2237 (GAA:c.[2237G>A], “2237G>A”) forming an immediate stop codon on the other (FIG. 1B). The mutations within GAA in this patient are ˜6.1 kb apart, and hence using a single double strand break (DSB) with homology directed repair from a long plasmid or viral donor would likely be inefficient. We therefore used a strategy utilizing two distinct Cas9 ribonucleoproteins (RNPs) with accompanying single stranded oligonucleotide (ssODN) templates encoding the gene correction. (FIG. 1B, Tables 1-2)

TABLE 1
PROTOSPACER AND RESPECTIVE PAMS USED FOR GENOMIC
TARGETING

| | | SEQ ID | |
|---|---|---|---|
| Name | Protospacer | NO: | PAM |
| 1441delT sgRNA | CTCGTTGTCCAGGTAGGCCC | 7 | GGG |
| 2237G > A sgRNA | TGGACCACCAGCTCCTGTAG | 8 | GGG |
| W746X sgRNA | GGACCACCAGCTCCTGTAGG | 60 | GGG |
| D645N sgRNA | GCCCAGGAAGCCGCAGACGT | 61 | TGG |
| R660H sgRNA | CAGAGGAGCTGTGTGTGCAC | 62 | TGG |

TABLE 2
ssODNs USED TO DIRECT HDR AFTER DSB FORMATION

| Name | Sequence (5′-3′) | SEQ ID NO: |
|---|---|---|
| 1441insT | CTTCCATGCAGGCCCTGGGTGGGGCCGGGTCTCCCCA | 9 |
| ssODN | CTGCAGCCTCTCGTTGTCCAGGTATGGCCCGGATCCA | |
| | CTGCCTTCCCCGACTTCACCAACCCC | |
| 2237A > G | TGCCCATCCCCCTTGCAGGTTCCCCAAGGACTCTAGC | 10 |
| ssODN | ACCTGGACTGTGGACCACCAGCTCCTGTGGGGAGAG | |
| | GCCCTGCTCATCACCCCAGTGCTCCAG | |
| W746X | TGCCCATCCCCCTTGCAGGTTCCCCAAGGACTCTAGC | 63 |
| ssODN | ACCTGGACTGTGGACCACCAGCTCCTGTGGGGAGAG | |
| | GCCCTGCTCATCACCCCAGTGCTCCAG | |
| D645N | AGAAATCCTGCAGTTTAACCTGCTGGGGGTGCCTCTG | 64 |
| ssODN | GTCGGGGCAGACGTCTGTGGCTTCCTGGGCAACACCT | |
| | CAGAGGAGCTGTGTGTGCACTGGACC | |
| R660H | GGGCCAACGTCTGCGGCTTCCTGGGCAACACCTCAGA | 65 |
| ssODN | GGAGCTATGTGTGCGCTGGACCCAGCTGGGGGCCTTC | |
| | TACCCCTTCATGCGGAACCACAACAG | |

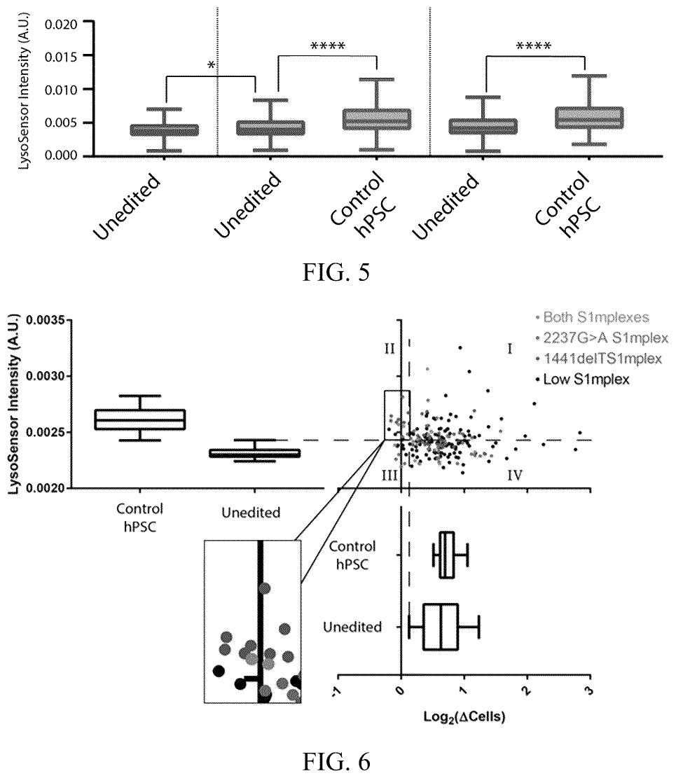

Specifically, using a combination of S1mplex and ArrayEdit technologies developed by our lab, we enriched for properly-edited iPSCs after delivery of the two genome editors by tracking the presence of genome editors within the nucleus (FIG. 1c). Using high-content analysis imaging of the iPSC clones during culture post delivery of the editors, we tracked the growth rate of clones, as well as screening the pH of the lysosome using a Lysosensor™ dye. Lysosensor™ is sensitive to the buildup of glycogen in the diseased lysosome of mutant GAA cells, as high glycogen neutralizes this otherwise acidic organelle (FIG. 1c; FIGS. 5-6). We isolated cell lines that were corrected at the 1441delT allele and the 2237G>A allele individually (FIG. 1d, termed ‘single corrected’). We also isolated a clone corrected at both GAA:c.[1441delT];[2237G>A] alleles (FIG. 1d, termed ‘double corrected’).

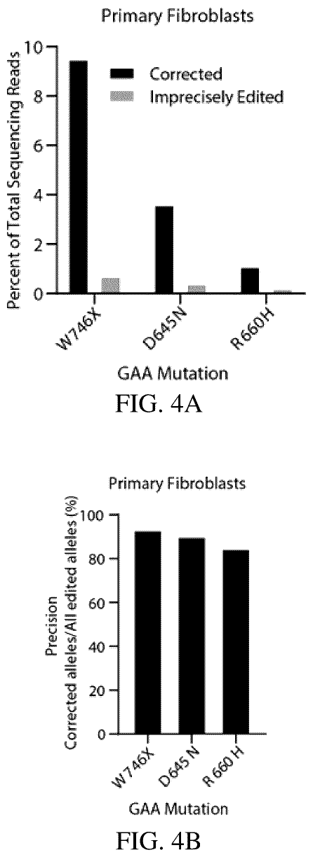

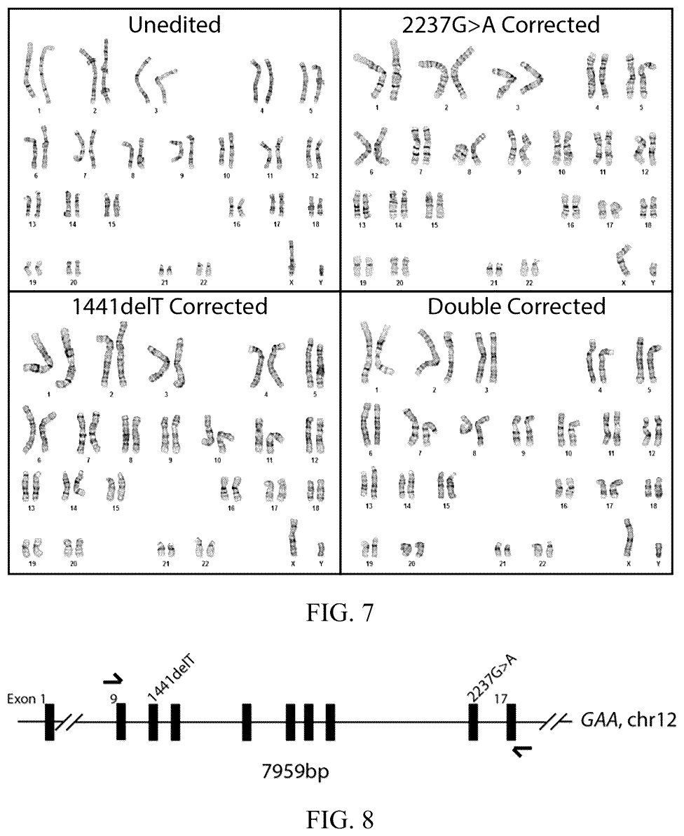

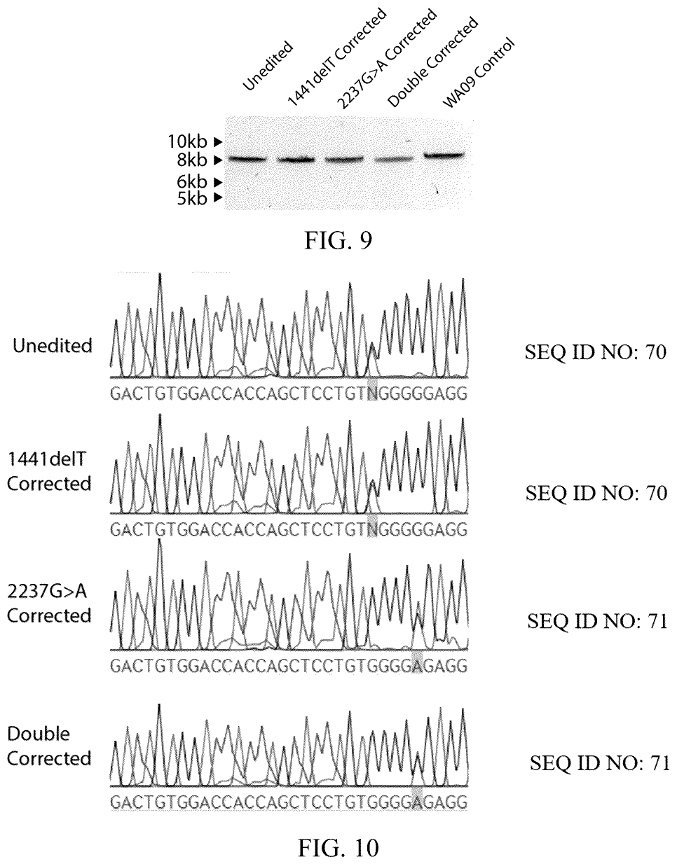

All gene-corrected lines remained pluripotent, and after karyotyping each of the isolated lines, we observed no large transversions or inversions (FIG. 7). Because genome editing can create large indel mutations, we also conducted an 8 kb PCR on GAA that included both sgRNA target sites and observed no genomic deletions between the sgRNA target sites. (FIG. 8, 9) Sequencing of these large PCR amplicons confirmed that both alleles were present and no other sequence abnormalities were detected at the edited loci. (FIG. 10) Finally, chromatograms from Sanger sequencing at the top three off-target sites for each sgRNA matched the untransfected, patient-derived cell line, indicating that none of the top off-target regions were modified by our editing strategy (Table 3). Similar S1mplex strategies generated edits at 84-93% precision at the sgRNA target in primary fibroblasts derived from other Pompe diseased patients (FIG. 4).

TABLE 3
OFF-TARGET SEQUENCING RESULTS

| Off- | | | |
|---|---|---|---|
| target | | | SEQ ID |
| site | Correction | Sequence | NO: |
| 2237 | Unedited | CCTCCCTTCCTAGACCACCAGCTCCTGCAGGAG | 72 |
| OT-1 | | GGCTTGG | |
| | 1441delT | CCTCCCTTCCTAGACCACCAGCTCCTGCAGGAG | 72 |
| | Corrected | GGCTTGG | |
| | 2237G > A | CCTCCCTTCCTAGACCACCAGCTCCTGCAGGAG | 72 |
| | Corrected | GGCTTGG | |
| | Double | CCTCCCTTCCTAGACCACCAGCTCCTGCAGGAG | 72 |
| | Corrected | GGCTTGG | |
| 2237 | Unedited | GCCCCTGCCTCTACAGGAGCAGGTGGTGAGGAT | 73 |
| OT-2 | | GGCTCCG | |
| | 1441delT | GCCCCTGCCTCTACAGGAGCAGGTGGTGAGGAT | 73 |
| | Corrected | GGCTCCG | |
| | 2237G > A | GCCCCTGCCTCTACAGGAGCAGGTGGTGAGGAT | 73 |
| | Corrected | GGCTCCG | |
| | Double | GCCCCTGCCTCTACAGGAGCAGGTGGTGAGGAT | 73 |
| | Corrected | GGCTCCG | |
| 2237 | Unedited | CACGCTGCCACGACAGGAGCTGCTGGTCAACTC | 74 |
| OT-3 | | CTCCTCT | |
| | 1441delT | CACGCTGCCACGACAGGAGCTGCTGGTCAACTC | 74 |
| | Corrected | CTCCTCT | |
| | 2237G > A | CACGCTGCCACGACAGGAGCTGCTGGTCAACTC | 74 |
| | Corrected | CTCCTCT | |
| | Double | CACGCTGCCACGACAGGAGCTGCTGGTCAACTC | 74 |
| | Corrected | CTCCTCT | |
| 1441 | Unedited | CCCCGTATCCCTGGTTGTCCAGGTGGGCCCTGG | 75 |
| OT-1 | | GAGAACA | |
| | 1441delT | CCCCGTATCCCTGGTTGTCCAGGTGGGCCCTGG | 75 |
| | Corrected | GAGAACA | |
| | 2237G > A | CCCCGTATCCCTGGTTGTCCAGGTGGGCCCTGG | 75 |
| | Corrected | GAGAACA | |
| | Double | CCCCGTATCCCTGGTTGTCCAGGTGGGCCCTGG | 75 |
| | Corrected | GAGAACA | |
| 1441 | Unedited | CAGCTGCCGTCTCGATGGCCAGGTAGGCCTGGG | 76 |
| OT-2 | | CAAGGAC | |
| | 1441delT | CAGCTGCCGTCTCGATGGCCAGGTAGGCCTGGG | 76 |
| | Corrected | CAAGGAC | |
| | 2237G > A | CAGCTGCCGTCTCGATGGCCAGGTAGGCCTGGG | 76 |
| | Corrected | CAAGGAC | |
| | Double | CAGCTGCCGTCTCGATGGCCAGGTAGGCCIGGG | 76 |
| | Corrected | CAAGGAC | |
| 1441 | Unedited | AATTAGGCTAGGGCCTACCTGGTCAATAATGAAATAAT | 77 |
| OT-3 | | TG | |
| | 1441delT | AATTAGGCTAGGGCCTACCTGGTCAATAATGAAATAAT | 77 |
| | Corrected | TG | |
| | 2237G > A | AATTAGGCTAGGGCCTACCTGGTCAATAATGAAATAAT | 77 |
| | Corrected | TG | |
| | Double | AATTAGGCTAGGGCCTACCTGGTCAATAATGAAATAAT | 77 |
| | Corrected | TG | |

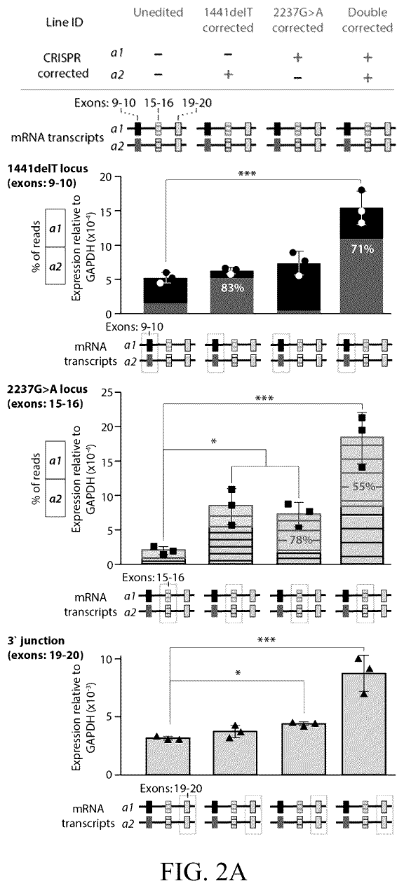

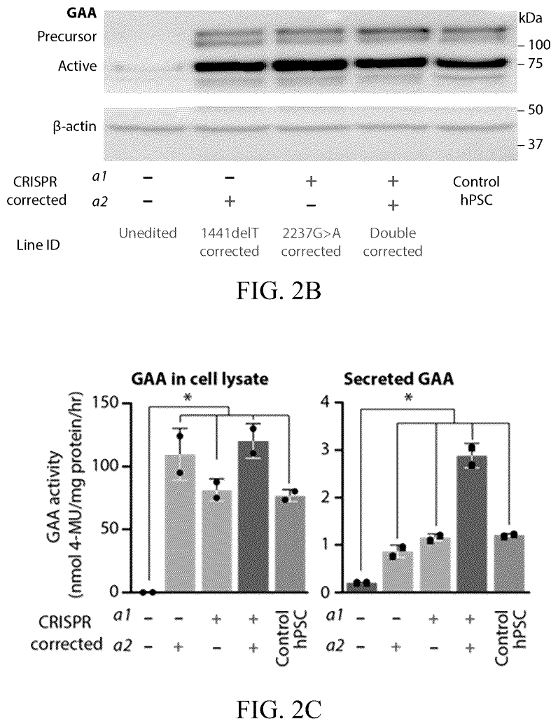

Quantitative RT-PCR (qRT-PCR) at the 3′ end of the GAA mRNA transcript as well as around each edited locus (FIG. 2a) indicated that the corrected loci were correctly expressed. We observed that the unedited line expressed the lowest levels of GAA when compared to internal GAPDH levels (FIG. 2a), despite the presence of full-length, mature mRNA that could be used to express protein (FIG. 2a). The single corrected lines also expressed mature mRNA, while the double corrected line contained approximately a 2-fold increase in GAA transcripts. We confirmed that double-corrected cell lines consistently produced greater amounts of mature mRNA than any other condition (FIG. 2a) by conducting qPCR at both edited loci.

By looking for the presence of disease variants and protoadjacent motif (PAM) wobbles introduced by the ssODN (FIG. 1B) via deep sequencing on endpoint PCR samples of mRNA, we observe that both alleles are expressed individually at higher levels (3-5 fold increase) than unedited cells (FIG. 2a). Each allele is expressed similarly to the corresponding single corrected line (FIG. 2a). These findings suggest nonsense mediated decay of the mutant transcript or cellular compensation to overcome the mutant allele within the single corrected lines. We detected active GAA protein using a Western blot (FIG. 2b) at levels comparable to a control hPSC line. We were also able to identify precursor polypeptides, which are important to protein secretion, showing the GAA transcripts from the edited alleles are being correctly translated and processed within cells. Notably, we are able to detect only small amounts of GAA protein and precursor polypeptides in the unedited iPSCs. All edited cell lines were able to secrete active GAA (FIG. 2c).

### Example 2: Enzymatic Cross-Correction by Gene-Corrected Cells

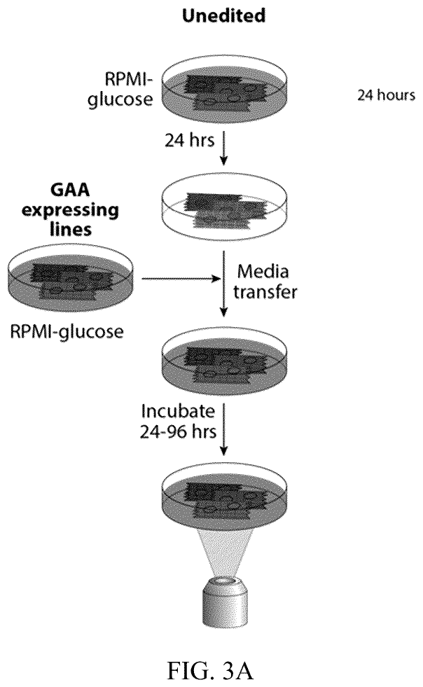

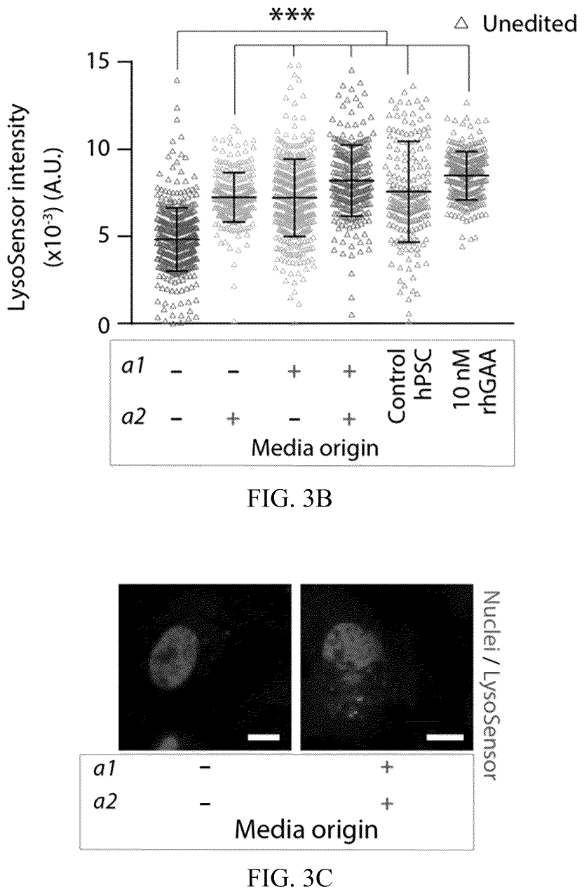

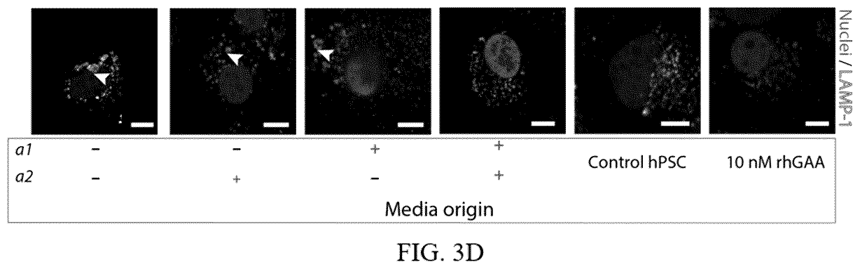

Detection of active GAA secretion led us to test the potential of edited cells to enzymatically cross-correct diseased cells (FIG. 3a). Because Pompe disease has a significant effect on cardiac tissue in infants, we differentiated all iPSC lines to cardiomyocytes (Pompe iPSC-CMs) using a previously described small-molecule inhibitor protocol (FIG. 3a). For all differentiations, we observed spontaneous contraction and confirmed the expression of α-actinin, a marker of cardiac lineage commitment. Similar to results seen in the iPSC state, differentiated corrected lines still expressed and secreted active GAA, as indicated in a 4-MUG cleavage assay on cardiomyocyte protein lysates and spent culture media. It has previously been demonstrated that by culturing in medium devoid of glucose, Pompe iPSC-CMs display an accumulation of glycogen within the lysosome³⁹. We performed a medium exchange experiment wherein we took partially spent, glucose-free medium from each corrected line (putatively containing secreted active GAA) and used it to replace glucose-free medium on unedited Pompe iPSC-CMs (FIG. 3a). One day after this media exchange, cells were stained with LysoSensor, and subsequent confocal microscopy was used to measure lysosome acidity as a proxy for glycogen clearance. As a control we added rhGAA to unedited Pompe iPSC-CMs to simulate ERT. When unedited cardiomyocytes were supplemented with 10 nM rhGAA (ERT), LysoSensor™ intensity increased, indicating a clearance of glycogen from the lysosome. Media from all edited cells were able to recover the lysosomal pH at 96 hr (FIG. 3b), and this clearance is expected to continue until normal levels of glycogen were reached. Within these cultures, lysosomal size of unedited Pompe iPSC-CMs in GAA-positive media was profiled through visualization of Lysosomal Associated Membrane Protein 1, (LAMP-1). In media from unedited cells, lysosomes were enlarged, consistent with buildup of glycogen (FIG. 3c). In comparison, when media was taken from double-corrected cells or supplemented with rhGAA, lysosomes appeared as punctae. Samples from single corrected cells fell between these two extremes. Taken together, the single- and double-corrected cells enzymatically cross-correct diseased cardiomyocytes quickly and effectively.

Based on our experimental results, cells corrected at both alleles a1 and a2 have been modeled to secrete 3-fold more GAA than those edited at a single allele. Gene correction rates relative to other potential editing outcomes come from experiments with patient-derived fibroblasts. After delivery of the S1mplex genome editors, approximately 80-90% of all edits achieved gene correction. We utilize the nomenclature of Shen et al to describe this ratio of gene correction to other editing outcome. The remaining 10-20% of edits are imprecise at the on-target site, which could destroy the PAM or modify the on-target site for subsequent editing of these alleles, and these alleles are tracked in silico.

Tables 4-6 provide the forward and reverse primer sequences (Table 4), the off-target sequences and corresponding genomic locus for each sgRNA used (Table 5), and the forward and reverse primers used to amplify off-target genomic loci (Table 6).

TABLE 4
FORWARD AND REVERSE PRIMERS FOR GENOMIC LOCI

| | | SEQ ID | | SEQ ID |
|---|---|---|---|---|
| Name | Primer F (5′-3′) | NO: | Primer R (5′-3′) | NO: |
| 1441delT | AGCTGCTCATTGACCT | 11 | CAATCCACATGCCGTCG | 12 |
| genomic | CCAG | | AAG | |
| 2237G > A | AATTCAGCCTCTTCCT | 13 | CATACGTTCCTCTTTCCG | 14 |
| genomic | GTGC | | CC | |
| Full length | TGACAGGTTTCCCTCT | 15 | TTGATAACCTACACTGCG | 16 |
| genomic | TCCCAG | | GGGG | |
| 1441delT | AGTGGGGCTTCCATGC | 17 | GGTTGGTGAAGTCGGGG | 18 |
| qPCR/NGS | AG | | AAG | |
| 2237G > A | CCAAGGACTCTAGCAC | 19 | GGGAAGTAGCCAGTCAC | 20 |
| qPCR/NGS | CTGGAC | | TTCGG | |
| W746X | TCCCATTCATCACCCG | 66 | AGGTCGTACCATGTGCCC | 67 |
| NGS | TATGC | | AA | |
| D645N | CTGAGGACCAGCCTG | 68 | CCACCCTACCAGACTGA | 69 |
| R660H | ACTCT | | GCA | |
| NGS | | | | |

TABLE 5
OFF-TARGET SEQUENCES AND CORRESPONDING GENOMIC LOCUS FOR
EACH sgRNA USED

| | Off-target sequence | | | |
|---|---|---|---|---|
| | | | SEQ ID | Chromosome |
| sgRNA Target Sequence | | | NO: | location |
| 2237G > A | OT1 | TAGACCACCAGCTCCTGCAG | 22 | chr8: −42696136 |
| TGGACCACCAGCTCCTGTAG | OT2 | CTCACCACCTGCTCCTGTAG | 23 | chr9: -123379574 |
| SEQ ID NO: 21 | OT3 | TTGACCAGCAGCTCCTGTCG | 24 | chr15: -77699091 |
| 1441delT | OT1 | CTGGTTGTCCAGGTGGGCCC | 26 | chr19: +9976610 |
| TGGACCACCAGCTCCTGTAG | OT2 | CTCGATGGCCAGGTAGGCCT | 27 | chr9: +113788274 |
| SEQ ID NO: 25 | OT3 | ATTATTGACCAGGTAGGCCC | 28 | chr20: -42195228 |

(MISMATCHES FROM PROTOSPACER ARE BOLDED)

TABLE 6
FORWARD AND REVERSE PRIMERS USED TO AMPLIFY OFF-TARGET
GENOMIC LOCI

| Off-Target | | SEQ ID | | SEQ ID |
|---|---|---|---|---|
| Primer | Primer F (5′-3′) | NO. | Primer R (5′-3′) | NO: |
| 2237-OT1 | CCCTCCTCTGTGTGCCATTA | 29 | GTGCCATATTTTGGGGACCAC | 30 |
| 2237-OT2 | GGGGCATGGTCAGATGATGG | 31 | CACAGAAATTCCTGAGGCCAAC | 32 |
| 2237-OT3 | GGAGAGGCTGACCTTCATGG | 33 | TCGTGCTTTCCTGACCATCG | 34 |
| 1441-OT1 | AGTGTGCTTCCACTGTCGTT | 35 | GTGCGGGTAACCTTCTCCAT | 36 |
| 1441-OT2 | TTCCTCTGCTGCTGAGTTGG | 37 | GCCGATTAAAAGGCTGTCGC | 38 |
| 1441-OT3 | AGAGCCCTGGAGGTCATTGT | 39 | CTGTCTGGCCTCTGAATCGG | 40 |

### Discussion

While potential off-target effects and other safety concerns have been extensively studied, the efficacy of genome editing strategies has yet to be quantitatively analyzed, especially in polygenic cases. Emerging human cell based and in silico models have been used to facilitate translation of gene augmentation therapy but have yet to be applied to genome editing. Our in vitro model constitutes a novel generalized framework to quantitatively understand the efficacy and potency of various genome editing strategies, and other gene and cell therapies.

We demonstrate biallelic gene correction with no detected off-target effects, and many of the common Pompe disease mutations can be targeted in an allele-specific manner using Sp.Cas9 strategies (Table 7). We observe that transcriptional regulation is driven by the endogenous promoter, potentially correcting a number of different isoforms for GAA. The targets for somatic cell genome editors therefore could expand from the traditional foci of liver and muscle to other tissues that may use alternate GAA isoforms. In contrast, in the gene augmentation approach, all cells must process a single isoform. Further, silencing from synthetic or viral elements has been observed for gene therapies, and in our hands with targeted knockin strategies that overexpress a transgene via a synthetic promoter. Transgene silencing raises concerns about the durability of viral gene therapies and proposed cell therapies where GAA is overexpressed from a safe harbor locus. In our strategy, post-translational processing of the enzyme also appears to be intact, as the distribution of processed GAA is identical to healthy controls. In contrast, GAA overexpression in mammalian cells can cause cellular stress, leading to differential trafficking and processing of the nascent translated peptide. The RNP-based correction strategy also avoids insertional oncogenesis by using a non-viral approach for delivery of the genome editor.

TABLE 7
SUBSET OF MUTATIONS IN GAA AND ACCOMPANYING ALLELE-SPECIFIC sgRNAs

| | | | Allele |
|---|---|---|---|
| | | SEQ ID | Frequency |
| Mutation | sgRNA Sequence | NO: | (×10⁻⁵) |
| c.118C > T | GAGGAGCCACTCAGCTCTCAGGG | 41 | 0.86 |
| c.258dupC | ATCGAAGCGGCTGTTGGGGGGGG | 42 | 2.65 |
| c.525delT | CTGGACGTGATGATGGAGAC- | 43 | 7.04 |
| | GAG | | |
| c.1822C > T | AGTGGCCGGCGTATCAGCCGTGG | 44 | 2.76 |
| c.1827delC | TGCTGGCCACGGCCGATA- | 45 | 3.75 |
| | GCCGG | | |
| c.1930_1936dupGCCGACG | AAGCCGCAGACGTCGGCCGTCGG | 46 | 1.17 |
| c.2242dupG | CACCAGCTCCTGTAGGGGGGAGG | 47 | 1.66 |
| c.2560C > T | ACCAAGGGTGGGGAGGCCTGAGG | 48 | 21.5 |
| c.2662G > T | TAACACGATCGTGAATTAGCTGG | 49 | 1.65 |

(BOLD DENOTES LOCATION OF MUTATIONS RELATIVE TO WILDTYPE FOR ALLELE-SPECIFIC SGRNAS. ITALICS DENOTES THE PAM SEQUENCE)

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of modifying a target gene ex vivo in a patient-derived cell, wherein the patient has an autosomal recessive disorder with compound heterozygous mutations, the method comprising
delivering to the cell a first modified guide RNA, a second modified guide RNA, a Cas9 polypeptide having nuclease activity, an avidin molecule, a first biotinylated donor polynucleotide, and a second biotinylated donor polynucleotide, wherein each modified guide RNA comprises,
a crRNA comprising, a single-stranded protospacer sequence and a first complementary strand of a binding region for the Cas9 polypeptide, and
a tracrRNA comprising a second complementary strand of the binding region for the Cas9 polypeptide,
wherein the crRNA or the tracrRNA comprises an aptamer that binds an avidin molecule,
wherein the crRNA and the tracrRNA hybridize through the first and second complementary strands of the binding region for the Cas9 polypeptide,
wherein the single-stranded protospacer sequence of the modified guide RNA hybridizes to a sequence in the target gene to be modified
wherein the first modified guide RNA and the first biotinylated donor polynucleotide correct a first diseased allele,
wherein the second modified guide RNA and the second biotinylated donor polynucleotide correct a second diseased allele.

2. The method of claim 1, wherein the patient-derived cell comprises an induced pluripotent stem cell, a progenitor cell, a mesenchymal stem cell, or a tissue-specific stem cell.

3. The method of claim 2, wherein the tissue-specific stem cell comprises a skeletal stem cell, a hematopoietic stem cell, an epithelial stem cell, or a neural stem cell.

4. The method of claim 1, wherein a first RNP complex comprises the first modified guide RNA, the Cas9 polypeptide, the avidin molecule and the first biotinylated donor polynucleotide; and a second RNP complex comprises the second modified guide RNA, the Cas9 polypeptide, the avidin molecule and the second biotinylated donor polynucleotide.

5. The method of claim 1, wherein the first modified guide RNA, the second modified guide RNA and the Cas9 polypeptide are expressed from one or more viral vectors.

6. The method of claim 5, wherein a first viral vector expresses the first modified guide RNA, and a second viral vector expresses the second modified guide RNA, and a third viral vector expresses the Cas9 polypeptide.

7. The method of claim 1, wherein the patient is a human patient and the autosomal recessive disorder is aspartylglucosaminuria, Batten disease, cystinosis, Fabry disease, Gaucher Disease Type I, II or III, Pompe Disease, Tay Sachs Disease, Sandhoff Disease, Metachromatic leukodystrophy, Mucolipidosis Type, I, II/III or IV, Hurler Disease, Hunter disease, Sanfilippo disease Types A,B,C,D, Morquio disease Types A and B, Maroteaux-Lamy disease, Sly disease, Niemann-Pick Disease Types A/B, C1 or C2, or Schindler Disease Types I or II.

8. The method of claim 1, wherein the wherein the avidin molecule is covalently linked to a donor polynucleotide, either directly or via a linker molecule.

9. The method of claim 1, wherein the biotinylated donor polynucleotide comprises a biotinylated nanoparticle, a dye, a contrast agent, a cell or tissue targeting ligand, or a peptide.

10. The method of claim 9, wherein the nanoparticle is a quantum dot, a gold particle, a magnetic particle, or a polymeric nanoparticle.

11. The method of claim 1, wherein the donor polynucleotide comprises single-stranded DNA, double-stranded DNA, RNA, or a duplex of RNA and DNA.

12. The method of claim 1, wherein the avidin molecule has one, two, three or four biotin binding sites, wherein the avidin molecule optionally comprises a fluorescent label.

13. A method of treating a patient with an autosomal recessive disorder with compound heterozygous mutations, comprising transplanting the cell made by the method of claim 1 into the subject.

14. A method of modifying a target gene in a patient-derived cell, wherein the patient has an autosomal recessive disorder with compound heterozygous mutations, the method comprising
delivering to the cell a first modified guide RNA, a second modified guide RNA, a Cas9 polypeptide having nuclease activity, an avidin molecule, a first biotinylated donor polynucleotide, and a second biotinylated donor polynucleotide, wherein each modified guide RNA comprises,
a crRNA comprising, a single-stranded protospacer sequence and a first complementary strand of a binding region for the Cas9 polypeptide, and
a tracrRNA comprising a second complementary strand of the binding region for the Cas9 polypeptide,
wherein the crRNA or the tracrRNA comprises an aptamer that binds an avidin molecule,
wherein the crRNA and the tracrRNA hybridize through the first and second complementary strands of the binding region for the Cas9 polypeptide,
wherein the single-stranded protospacer sequence of the modified guide RNA hybridizes to a sequence in the target gene to be modified
wherein the first modified guide RNA and the first biotinylated donor polynucleotide correct a first diseased allele,
wherein the second modified guide RNA and the second biotinylated donor polynucleotide correct a second diseased allele.

15. The method of claim 14, wherein a first RNP complex comprises the first modified guide RNA, the Cas9 polypeptide, the avidin molecule and the first biotinylated donor polynucleotide; and a second RNP complex comprises the second modified guide RNA, the Cas9 polypeptide, the avidin molecule and the second biotinylated donor polynucleotide.

16. The method of claim 14, wherein the first modified guide RNA, the second modified guide RNA and the Cas9 polypeptide are expressed from one or more viral vectors.

17. The method of claim 16, wherein a first viral vector expresses the first modified guide RNA, and a second viral vector expresses the second modified guide RNA, and a third viral vector expresses the Cas9 polypeptide.

18. The method of claim 14, wherein the patient is a human patient and the autosomal recessive disorder is aspartylglucosaminuria, Batten disease, cystinosis, Fabry disease, Gaucher Disease Type I, II or III, Pompe Disease, Tay Sachs Disease, Sandhoff Disease, Metachromatic leukodystrophy, Mucolipidosis Type, I, II/III or IV, Hurler Disease, Hunter disease, Sanfilippo disease Types A,B,C,D, Morquio disease Types A and B, Maroteaux-Lamy disease, Sly disease, Niemann-Pick Disease Types A/B, C1 or C2, or Schindler Disease Types I or II.

19. The method of claim 14, wherein the wherein the avidin molecule is covalently linked to a donor polynucleotide, either directly or via a linker molecule.

20. The method of claim 14, wherein the biotinylated donor polynucleotide comprises a biotinylated nanoparticle, a dye, a contrast agent, a cell or tissue targeting ligand, or a peptide.

21. The method of claim 20, wherein the nanoparticle is a quantum dot, a gold particle, a magnetic particle, or a polymeric nanoparticle.

22. The method of claim 14, wherein the donor polynucleotide comprises single-stranded DNA, double-stranded DNA, RNA, or a duplex of RNA and DNA.

23. The method of claim 14, wherein the avidin molecule has one, two, three or four biotin binding sites, wherein the avidin molecule optionally comprises a fluorescent label.
